(19) United States
(12) Patent Application Publication
(10) Pub. No.: US 2026/0306707 A1
(43) Pub. Date: Oct. 8, 2026

(54) DOUBLE NETWORKED 3D-PRINTED BIOMATERIALS

(71) Applicant: THE BRIGHAM AND WOMEN'S HOSPITAL, INC., Boston, MA (US)

(72) Inventors: Yu Shrike Zhang, Boston, MA (US); Xiao Kuang, Boston, MA (US)

(21) Appl. No.: 18/998,473

(22) PCT Filed: Jul. 27, 2023
(86) PCT No.: PCT/US2023/028853

§ 371 Date: Jan. 25, 2025

Related U.S. Application Data
(60) Provisional application No. 63/392,792, filed on Jul. 27, 2022.
(60) Provisional application No. 63/451,633, filed on Mar. 12, 2023.

(51) Int. Cl. A61L 27/22; A61F 2/06; A61L 27/16; A61L 27/18; A61L 27/20; A61L 27/36; A61L 27/52; B29C 64/118; B29C 64/30; B29K 75/00; B29K 105/00; B29L 31/00; B33Y 10/00; B33Y 40/20; B33Y 70/10; B33Y 80/00; G01N 33/50

(52) U.S. Cl. CPC A61L 27/222; A61F 2/062; A61L 27/16; A61L 27/18; A61L 27/20; A61L 27/3604; A61L 27/52; B29C 64/118; B29C 64/30; B33Y 10/00; B33Y 40/20; B33Y 70/10; B33Y 80/00; G01N 33/5082; A61L 2430/22; B29K 2005/00; B29K 2075/00; B29K 2089/00; B29K 2105/0035; B29K 2105/0061; B29K 2105/0088; B29L 2031/7532

(57) ABSTRACT

A 3D-printed biomaterial is described that comprises a double-networked hydrogel comprising gelatin or gelatin methacryloyl and alginate, and cells. Methods of making the 3D-printed biomaterial by extruding a hydrogel comprising gelatin or gelatin methacryloyl and alginate from a 3D-printer and converting the hydrogel to a double-networked hydrogel by cross-linking the gelatin with a first cross-linking method and cross-linking the alginate with a second cross-linking method are also described. Methods of treating a damaged or diseased blood vessel and testing vasoactive drugs using 3D-printed blood vessels are also described.

### CONTINUING APPLICATION DATA

This application claims the benefit of U.S. Provisional Application Ser. No. 63/392,792, filed Jul. 27, 2022, and U.S. Provisional Application Ser. No. 63/451,633, filed Mar. 12, 2023, the disclosures of which are incorporated by reference herein.

### STATEMENT REGARDING FEDERALLY SPONSORED RESEARCH

This invention was made with government support under Grant Number R01HL153857, awarded by the National Institutes of Health. The Government has certain rights in this invention.

### BACKGROUND

Biofabrication is capable of manufacturing anatomical and tissue models functional tissues and using living cells and extracellular matrix (ECM)-like biomaterials, finding applications in diverse areas such as tissue engineering and regenerative medicine. To design biomaterials for biofabricating physiologically relevant engineered tissues, it is necessary to recapitulate the hierarchal structures, good mechanical properties, and essential bioactivities of ECM, as well as good processability to replicate the three-dimensional (3D) anatomical geometries. ECM provides structural and functional support roles for tissues and life at different length scales. For example, most native soft tissues, such as blood vessels, reveal excellent mechanical properties, such as high toughness (>200 J m⁻²), to sustain physiological deformation. This high fracture toughness of soft tissues is mainly associated with the multiscale hierarchical structures of strong collagen fibrils and intertwined highly hydrated proteoglycan. Black et al., Biophys. J. 94, 1916-1929 (2008). The stiff collagen fibril network acts as the loadbearing component while providing cell-anchoring sites; the soft-phase of proteoglycan featuring low stiffness facilitates nutrition transport and cell signaling, all contributing to accommodating the residing cells. Hydrogels, consisting of highly hydrated polymer networks with high water content (>70%), have become the prime candidate to support the spatiotemporal activities of encapsulated cells through biofabrication. Y. S. Zhang, A. Khademhosseini, Science 356, eaaf3627 (2017). In the past decades, tremendous advances in engineered hydrogels using synthetic and natural polymers with different crosslinking methods have allowed sophisticated control over hydrogels' physicochemical properties and bioactivities for biofabrication. Gao et al., Adv. Mater., 2006362 (2021).

Conventional hydrogels are designed with loose crosslinks and large network mesh sizes to facilitate cellular behaviors; however, they significantly compromise mechanical robustness. Fuchs et al., Adv. healthcare Mater. 9, 1901396 (2020). In pursuit of enhanced mechanical properties, tough hydrogels, such as double-network (DN) hydrogels and hydrogel composites, have been developed through novel hydrogel-network design and structural engineering approaches. Zhao et al., Chem. Rev. 121, 4309-4372 (2021). These extreme-mechanical tough hydrogels find diverse biomedical applications. Yuk et al., Nature 575, 169-174 (2019). However, most of these reported formulations are not benign to cell encapsulation. Additionally, the resulting tough hydrogels with dense polymer networks substantially restrict the behaviors of embedded cells, such as their spreading. Therefore, the only possibility of these tough hydrogels interacting with cells is to post-seed on the surfaces, which is not physiological. Hong et al., Adv. Mater. 27, 4035-4040 (2015). Meanwhile, porous hydrogels have been fabricated using sacrificial liquid or solid templates to accommodate cellular performances. Thorson et al., Acta Biomater. 94, 173-182 (2019). Recently, aqueous two-phase emulsions (ATPEs), i.e., liquid-liquid phase-separation of two immiscible water-soluble components, have been exploited to create microporous biomaterial hydrogels with enhanced cellular functions. Wang et al., Adv. Mater., 2107038 (2021). However, state-of-the-art porous hydrogels including the ones formed from ATPEs exhibit weak mechanical properties (tensile strength<50 kPa, toughness<50 J m⁻²) far from native tissues. Therefore, the design of advanced biomaterial hydrogels that exhibit high toughness and strength competing with native soft tissues, that simultaneously support favorable cellular activities, and that are suitable for biofabrication of physiologically relevant tissues remains an unmet challenge.

### SUMMARY

The mechanical properties of soft tissues, including toughness and strength, play a critical role in supporting organ functions. The inventors have developed hierarchical tough hydrogel systems that can self-assemble into bi-continuous microstructures and solidify into heterogeneous tough hydrogels. The tough biomaterial hydrogels can be engineered with load-bearing hard regions and cell-instructive soft regions at the microscale. The protein-based hydrogel-precursors can be mixed with living cells as bioinks for various bioprinting techniques. Using this method, complex anatomical living tissues can be directly fabricated. The tough hydrogels act as cell-benign and biodegradable scaffolds and accelerate tissue formation without sacrificing excellent mechanical properties.

Three-dimensional (3D) bioprinting of vascular tissues that are mechanically and functionally comparable to their native counterparts is an unmet challenge. The inventors have developed a tough double-network hydrogel (bio)ink for microfluidic (bio)printing of mono- and dual-layered hollow conduits to recreate vein- and artery-like tissues, respectively. The tough hydrogel consisted of energy-dissipative ionically crosslinked alginate and elastic enzyme-crosslinked gelatin. The 3D-bioprinted venous and arterial conduits exhibited key functionalities of respective vessels including relevant mechanical properties, perfusability, barrier performance, expressions of specific markers, and susceptibility to severe acute respiratory syndrome coronavirus-2 pseudoviral infection. Notably, the arterial conduits revealed physiological vasoconstriction and vasodilatation responses. The feasibility of these conduits for vascular anastomosis was also explored. Altogether, biofabrication of mechanically and functionally relevant vascular conduits are presented, showcasing their potentials as vascular models for disease studies in vitro and as grafts for vascular surgeries in vivo, possibly serving broad biomedical applications in the future.

### BRIEF DESCRIPTION OF THE FIGURES

FIGS. 1A-1J provide schemes, graphs, and images showing the design of bi-continuous tough hydrogels for biofabrication. (A) Scheme of using AVPS bioinks in biofabrication of bi-continuous tough hydrogel-based physiologically relevant tissues; (B) Scheme of a proteoglycan-like biopolymer and immiscible template-polymer for constructing AVPS emulsions. (C) Complex viscosity versus shear-frequency for 12 wt % Alg-Gel and PVA (300 kDa) at various temperatures. (D) Phase diagram of Alg-Gel/PVA emulsion. Feeding: pink marks; Alg-Gel-rich phase nodes: orange marks; Alg-Gel-poor phase nodes: blue marks; Fitted bimodal-curve: green solid-line; tie-line: dark dash-line. Inset shows a confocal fluorescence micrograph of bi-continuous microstructures of an AVPS emulsion. (E) Color-contour of the Alg-Gel-poor phase mass ratio for Alg-Gel/PVA emulsion. (F) Average biopolymer-rich phase size versus annealing time for Gel/PVA (6/4) and Alg-Gel/PVA (6/4) emulsions at 34° C. (n>20). Inset showing the characteristic size of modeled bi-continuous microstructures. (G-H) Coaxially (bio)printed tube: photograph (G) and fluorescence micrograph (H). (I-J) A volumetrically (bio)printed meniscus: photograph (I) and fluorescence micrograph (J). Data are mean values±SDs. Scale bars, 200 μm (D, H, J); 2 mm (G, I).

FIGS. 2A-2I provide graphs and schemes showing the mechanical properties of bi-continuous tough hydrogels. (A) Schematic of multiscale hierarchical structures of BC-DN hydrogels. Microscale interconnected soft- and hard-phases of hydrogels consisted of distinct network-mesh sizes. (B) Stress-strain curves of DN hydrogels of various mass concentrations as labeled. (C) Fracture energy and toughness values for DN hydrogels of various mass concentrations (n=3). Inset shows the snapshots of the critical strain by pulling a notched DN hydrogel (12 wt % Alg-Gel) until crack propagation. (D) Compressive moduli and compressive stresses at 85% strain versus mass concentrations for Alg-Gel DN hydrogels (n=3). (E) Stress-strain curves of unnotched and notched DN hydrogels (6 wt % Alg-Gel) and BC-DN hydrogels of various Alg-Gel concentrations (6-10 wt %). (F) Young's moduli, tensile strengths, and fracture energies for DN and BC-DN hydrogels in (E) (n=3). (G) FEA of a BC-DN hydrogel using 2D bi-continuous model under uniaxial stretching. (H-I) Ashby diagrams of tensile strength versus tensile strain (H) and fracture energy versus tensile strain (I) for Alg-Gel-based DN and BC-DN hydrogels, other reported biomaterial hydrogels, and soft tissues. Data are mean values±SDs. Scale bar, 2 mm.

FIGS. 3A-3J provide graphs and images showing the cellular activities in cell-laden tough hydrogels. (A) Fluorescence micrographs showing cell-tracker-labeled NIH/3T3 cells (green) in hydrogel matrices (red) taken at 16 hours of culture. (B) Cell density in cell-laden hydrogels at 0 and 16 hours of culture (n=3). (C) Compressive moduli for DN and BC-DN hydrogels (n=3). Soft- and hard-hydrogels were obtained by crosslinking biopolymer-rich and biopolymer-poor phase aliquots from the BC-DN emulsion after complete phase-separation. (D) Effective diffusion coefficients for various hydrogels in (C) (n=3). (E) Confocal fluorescence micrographs showing F-actin staining of hMSCs encapsulated in DN and BC-DN hydrogels on day 7. F-actin: green; nucleus: blue. (F) Fluorescence micrograph of F-actin-stained hMSCs in red-fluorescent BC-DN hydrogel on day 14. Red: hydrogel; F-actin: green; nucleus: blue. (G) Cell metabolic activities for hMSCs quantified by cell proliferation assay (n=3). (H) Average cell lengths in hMSC-laden BC-DN hydrogels plotted against culture time (n=50-100). (I) Confocal fluorescence micrographs showing F-actin staining of NIH/3T3 fibroblasts, HUVSMCs, and HUVECs separately encapsulated in BC-DN hydrogels on day 7. F-actin: green; nucleus: blue. (J) Cell metabolic activities for different cell types in (I) (n=3). Data are mean values±SDs. ns: no significant difference, *p<0.05, **p<0.01, ***p<0.001. Scale bars, 200 μm.

FIGS. 4A-4I provide graphs and images showing biofabrication of tough hydrogel-based neo-cartilage. (A) Scheme of meniscal cartilage. (B) Timeline of biofabrication of engineered neo-cartilage and their subsequent culture protocols. (C-D) Confocal fluorescence micrographs showing co-staining of F-actin staining and collagen X (C) and aggrecan staining (D) for hMSC-laden BC-DN hydrogels on day 14 of chondrogenesis. F-actin: green; nucleus: blue in (C). Aggrecan: green; collagen: red; nucleus: blue in (D). (E) Relative expression level of representative chondrogenic genes (SOX9, AGCAN, COL1A1, COL10A1, COMP, and ELASTIN) for hMSC-laden BC-DN hydrogels (n=2-3). Results were normalized by the housekeeping gene (GAPDH) and comparison were referenced to day 7. (F) Histological images of hMSC-laden BC-DN hydrogels: (i) Masson's trichrome for collagen deposition; (ii) Alcian blue for aggrecan's deposition. Black arrow: collagen in soft-phase; Orange arrow: collagen at soft/hard-phase interfaces; Gren arrow: aggrecan in soft-phase. (G) Young's moduli and compressive stresses at 80% strain for hMSC-laden BC-DN hydrogels on different periods after differentiation (n=2-3). (H) Compressive stress-strain curves of pristine and boosted hMSC-laden BC-DN hydrogels on day 14 of chondrogenesis. Inset snapshots illustrate the compression of a neo-cartilage. (I) Confocal fluorescence micrographs showing F-actin staining of engineered neo-cartilages: pristine (i) and after compression fracture (ii). Data are mean values±SDs. ns: no significant difference, *p<0.05, **p<0.01, and ***p<0.001. Scale bars: 50 μm (I), 200 μm (C, D, F), 2 mm (H).

FIGS. 5A-5I provide schemes, graphs, and images showing coaxially bioprinted tough hydrogels-based vascular conduits. (A) Scheme of blood vessels consisting of a smooth muscle layer and endothelial layer. (B) Timeline of biofabrication of engineered vessels and their subsequent culture protocols. Cell tubes were coaxially bioprinted using HUVSMC-laden BC-DN emulsion bioink. (C) Fluorescence micrographs showing live/dead staining of printed cell tubes on day 7 post-printing. Live: green; dead: red. (D) Confocal fluorescence micrographs showing F-actin staining of cell tubes on day 14: lateral view (i) and cross-section view (ii). The cell-laden tubes were boosted on day 7 by soaking in 3 w/v % CaCl₂. F-actin: green; nucleus: blue. (E) Tensile stress-strain curves for pristine and boosted cell-laden tubes at various culture periods as labeled. (F) Confocal fluorescence micrographs showing immunostaining of the engineered vessels on day 28. HUVECs were post-seeded in the lumen on day 21. α-SMA: green; VE-cadherin: red; nucleus: blue. (G) Schematics of suture anastomosis (i) and experimental stretching of anastomosed conduits by human vein and (bio)printed vessels up to 60% strain (ii). (H) Suture-retention strength of native veins and (bio)printed vessels (n=3-5). (I) cyclic load-unloading test for anastomosed conduits with a maximum strain of 20%. Data are mean values±SDs. Scale bars, 400 μm (C, D, F), 4 mm (G).

FIGS. 6A-6G provide graphs and images showing the design and mechanical properties of tough DN hydrogel (bio)inks. (A) Schematics of DN hydrogel containing physically crosslinked alginate by calcium as the first network and chemically crosslinked gelatin by mTG as the second network. (B) Apparent viscosities as a function of shear rate on (bio)ink (MAlg1Gel15) and its individual components (MAlg1 and Gel15) at 37° C. (C) Loading-unloading tensile stress-strain curves of MAlg1, Gel15, and MAlg1Gel15 hydrogels crosslinked by CaCl₂, mTG, and CaCl₂/mTG, respectively. The maximum strain was 25%. (D) Loading-unloading tensile stress-strain curves of the MAlg1Gel15 hydrogels with four different crosslinking and post-treatment methods: CaCl₂), mTG, CaCl₂)/mTG, and CaCl₂/mTG/EDTA. The maximum strain was 25%. Tensile stress-strain curves (E), Young's moduli (F), and fracture energies (G) of MAlg1Gel15 hydrogels with four different crosslinking and post-treatment methods: CaCl₂, mTG, CaCl₂/mTG, and CaCl₂)/mTG/EDTA. ns: no significant difference, **p<0.01, ***p<0.001, ****p<0.0001.

FIGS. 7A-7M provide graphs and images showing microfluidic extrusion (bio)printing and mechanical properties of tubular conduits. (A) Schematics of microfluidic extrusion (bio)printing of mono-layered and dual layered vascular conduits. (B) Temperature-dependent viscosities of the alginate-gelatin and the alginate-GelMA hybrid (bio)inks. (C) Shear storage moduli (blue) and loss moduli (red) as a function of shear stress on the alginate-gelatin and the alginate-GelMA hybrid (bio)inks. (D) Representative lateral-view bright-field images (i, ii) and fluorescence microscopic images (iii, iv), as well as cross-sectional-view fluorescence microscopic images (v, vi) of mono-layered (top) and dual-layered (bottom) hollow tubes. Scale bars, 200 μm. (E) Fluorescence microscopic images of (bio)printed hollow conduits (i, an HMS shaped tube; ii, an MIT-shaped tube; iii, a randomly placed long tube). Scale bars, 1 cm. (F) Quantitative evaluations of high-throughput (bio)printing of tubes using 1 mL of (bio)inks. Comparisons of mechanical properties of (bio)printed vascular tubes using different (bio)inks and mouse vena cave: tensile stress-strain curves (G), tensile strengths (H), Young's moduli (I), and failure strains (J). ns: no significant difference, *p<0.05, ***p<0.001, ****p<0.0001. (K) Photographs of tensile tests for (bio)printed mono-layered tubes and mouse vena cave. (L) Comparisons of burst pressures for (bio)printed tubes with different sizes and compositions as well as native vessels. ns: no significant difference, *p<0.05, ****p<0.0001. (M) Photographs of (bio)printed mono-layered tubes before and after inflation during burst pressure test. Scale bars, 1 cm.

FIGS. 8A-8G provide graphs and images showing structural and biological functions of (bio)printed veinous conduits. (A) Schematics showing structures of the native vein and 3D-(bio)printed venous conduit. (B) Fluorescence microscopic images showing the viability of HUVSMCs and HUVECs at different time points of culture. Green, live cells; red, dead cells. Scale bar, 200 μm. (C) Quantified viability of HUVSMCs and HUVECs at indicated time points. ns: no significant difference. (D) Fluorescence confocal images of the immunostained venous conduits exhibiting expressions of F-actin by both HUVSMCs and HUVECs, ZO-1 by HUVECs, and α-SMA by HUVSMCs, for cell separately cultured. The cells were counterstained with DAPI for nuclei. Scale bars, 100 μm. (E) Fluorescence confocal images of the immunostained venous conduits exhibiting expressions of ZO-1 by HUVECs and α-SMA by HUVSMCs, for cell co-cultured. The cells were counterstained with DAPI for nuclei. Scale bars, 100 μm. (F) Diffusion of 3-5-kDa FITC-Dex in (bio)printed mono-layered conduits in the absence (top) and presence (bottom) of endothelium formed by HUVECs in the lumens. Scale bar, 200 μm. (G) Quantified 3-5-kDa FITC-Dex diffusion ratio changes in the absence and presence of endothelium formed by HUVECs in the lumens of mono layered conduits.

FIGS. 9A-9H provide graphs and images showing structural and biological functions of (bio)printed arterial conduits. (A) Schematics showing structures of the native artery and (bio)printed arterial conduit. (B) Fluorescence microscopic images showing the viability of (bio)printed HUASMCs at different time points of culture. Green, live cells, red, dead cells. Scale bars, 100 μm. (C) Fluorescence confocal images of the immunostained artery exhibiting expressions of F-actin by HUASMCs. The cells were counterstained with DAPI for nuclei. Red, F-actin; blue, nuclei. Scale bars, 100 μm. (D) Fluorescence confocal images of the immunostained artery exhibiting expressions of ZO-1 by HUAECs and α-SMA by HUASMCs. The cells were counterstained with DAPI for nuclei. Red, ZO-1; green, α-SMA; blue, nuclei. Scale bars, 100-μm. (E) Diffusion of 3-5-kDa FITC-Dex in the (bio)printed dual-layered conduits in the absence (top) and presence (bottom) of endothelium formed by HUAECs in the lumens. Scale bar, 200 μm. (F) Fluorescence confocal images of the immunostained artery exhibiting expressions of α-1a adrenergic receptor and muscarinic acetylcholine receptor by HUASMCs. The cells were counterstained with DAPI for nuclei. Red, α-1a adrenergic receptor; green, muscarinic acetylcholine receptor; blue, nuclei. Scale bars, 100 μm. (G) Vasoactivities of (bio)printed arterial conduits in response to phenylephrine and acetylcholine. Scale bar, 200 μm. (H) Quantified changes in diameter of bioprinted arterial conduits in response to phenylephrine and acetylcholine.

FIGS. 10A-10F provide graphs and images showing in vitro, ex vivo, and in vivo applications of (bio)printed vascular conduits. (A) Fluorescence confocal images showing the expression of ACE2 receptors by HUVSMCs and HUVECS in the bioprinted vascular conduits. The cells were counterstained with DAPI for nuclei. Red, ACE2 receptors; blue, nuclei. Scale bars, 100 μm. (B) Fluorescence and bright-field microscopic images showing mcherry-expressing pCoV-VPs-infected HUVSMCs and HUVECs in the bioprinted vascular conduits in the absence of antiviral drug (pCoV-VPs) and in the presence of 10-μM remdesivir (pCoV-VPs+RDV) or 10-μM amodiaquine (pCoV-VPs+ADQ). Scale bars, 100 μm. (C) Fluorescence microscopic images showing live/dead staining of cells in the bioprinted vascular conduits after pCoV-VP infection without antiviral drugs (pCoV-VPs) and with 10-μM remdesivir (pCoV-VPs+RDV) or 10-μM amodiaquine (pCoV-VPs+ADQ). Green, live cells; red, dead cells. Scale bars, 100 μm. (D) MTS assay showing metabolic activities of cells in the bioprinted vascular conduits after pCoV-VP infection in the absence and presence of antiviral drugs. *p<0.05. (E) Ex vivo connection and perfusion of bioprinted vascular conduits linked to native vessels by bioglue. i, ii, a small-sized printed vascular conduit (1-mm outer-diameter) anastomosed with a mouse aorta; iii, iv, a printed larger-sized (5-mm outer-diameter) vascular conduit anastomosed with a human popliteal vein. Scale bars, 20 mm. (F) In vivo implant and perfusion between mouse vena cava and a printed vascular conduit in mouse. i, schematic diagram showing in vivo connection between mouse vena cava and a bioprinted vascular conduit; ii, exposure of mouse vena cava; iii, perfusion of the blood after anastomosis. Scale bar, 1 mm.

FIGS. 11A and 11B provide cross-sectional images of the inner and outer layer of double layer tubes including polyurethane.

FIG. 12 provides a graph of the strain-force curve of 3D-printed hollow tubes including polyurethane.

### DETAILED DESCRIPTION

A 3D-printed biomaterial is provided that comprises a double-networked hydrogel comprising gelatin and alginate, and cells. Methods of making the 3D-printed biomaterial by extruding a hydrogel comprising gelatin and alginate from a 3D-printer and converting the hydrogel to a double-networked hydrogel by cross-linking the gelatin with a first cross-linking method and cross-linking the alginate with a second cross-linking method are also provided. Methods of treating a damaged or diseased blood vessel and testing vasoactive drugs using 3D-printed blood vessels are also provided.

### Definitions

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. For example, as used in this specification and the appended claims, the singular forms “a”, “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form. The conjunctive phrase “and/or” indicates that either or both of the items referred to can be present.

The phrase “consisting essentially of” means that the composition or method may include additional ingredients and/or steps, but only if the additional ingredients and/or steps do not materially alter the basic and novel characteristics of the claimed composition or method.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

As used herein, the term “about” means±10% of the recited value.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

As used herein, the terms “printing” and “bioprinting” are used interchangeably, and cover both normal 3D printing and printing that includes cells in the bioink.

A weight percent (wt. %) of a component, unless specifically stated to the contrary, is based on the total weight of the formulation or composition in which the component is included. Furthermore, where the weigh percentage of the polymeric components of a hydrogel are provided, it can be assumed that the remaining weight percentage consists of water, cells, and other minor additives that may be present in the hydrogel unless the percentages of other components are specifically provided.

“Biocompatible” as used herein, refers to the capability of a material to be integrated into a biological system without harming or being rejected by the system. Examples of harm include inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

The term polymer, as used herein, also refers to polymer precursors that are cross-linked to form the polymer, unless it is specifically indicated that the polymers do not include polymer precursors.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

### Double-Networked 3D-Printed Biomaterials

In one aspect, the present invention provides a 3D-printed biomaterial comprising a double-networked hydrogel comprising gelatin (e.g., gelatin methacryloyl (GelMA)) and alginate, and cells. Hydrogels are water-rich polymers that can hold considerable amounts of water and are usually benign to embedded cells. Hydrogels are polymeric networks with hydrophilic chains crosslinked either covalently or physically (via intra- and intermolecular attractions).

A double-networked hydrogel is one in which two different cross-linking networks exist in the hydrogel polymer, forming a double-crosslinked copolymer. Double cross-linking can also be referred to as biorthogonal cross-linking. For example, a double-networked hydrogel can be one in which one polymer is photocrosslinked, and another, second polymer is chemically cross-linked. Alternately, a double-networked polymer can be one in which different chemical methods are used to create two different cross-linking networks. For example, a double-networked polymer can be one in which a first polymer such as alginate is physically (e.g., ionically) cross-linked by calcium (e.g., CaCl₂) while a second polymer such as gelatin is covalently cross-linked using microbial transglutaminase (mTG). Alginate and GelMA are both biocompatible polymers.

In some embodiments, the hydrogel comprises at least two different types of material. These materials can form at least two layers, or the materials can intermixed but discrete, such as being in an emulsion. Including two different materials allows the biomaterial including the hydrogel to exhibit the advantages of these different materials. For example, it can allow the hydrogel to provide regions that supports cell growth, while also providing regions that provide structural support for the hydrogel. For example, in some embodiments one material is mechanically stronger, while the other is mechanically weaker but with a more open pore structure.

In some embodiments, the hydrogel comprises a stronger layer having a higher stiffness and a smaller mesh size, and a weaker layer having a lower stiffness and a larger mesh size. Stronger and weaker layers refer to layers having stronger or weaker mechanical strength, relative to the other layer. The stronger layer can also be referred to as a hard layer, while the weaker layer can be referred to as a soft layer. In some embodiments, the weaker/soft layer comprises cells.

The strength, or stiffness, of the hydrogel regions can be represents by their Young's modulus, which is the ratio of stress to elastic strain. In some embodiments, the stronger layer has a Young's modulus from about 50 to about 1000 kPa. In further embodiments, the stronger layer has a Young's modulus from about 100 to about 700 kPa. In further embodiments, the stronger layer has a Young's modulus from about 200 to about 500 kPa. In additional embodiments, the weaker layer has a Young's modulus from about 0.1 to about 45 kPa, while in further embodiments the weaker layer has a Young's modulus from about 0.1 to about 10 KPa, from about 0.1 to about 1 kPa, or from about 0.5 to about 5 kPa. The Young's modulus can be determined using methods known to those skilled in the art, such as the use of a texture analyzer.

The hydrogel can include regions having different mesh sizes. The stronger layer typically has a smaller mesh size, while the weaker layer typically has a larger mesh size. Mesh size (ξ) is the average distance between two neighboring network junctions that are connected by a polymer chain in a hydrogel. The mesh size can readily be determined by those skilled in the art. See Sorichetti et al., Macromolecules, 53, 7, 2568-2581 (2020).

The strength of the hydrogel regions can be changed by varying the polymer composition of that region. In some embodiments, the weaker layer comprising 1% to 3% alginate by weight and 2% to 4% GelMA by weight, or 1% to 5% alginate by weight and 1% to 3% GelMA. In some embodiments, the stronger layer comprising 0.5% to 2% alginate by weight and 10% to 20% GelMA by weight, or 0.1% to 3% alginate by weight and 15% to 25% GelMA by weight.

Other characteristics that can be important to 3D-printed biomaterials include toughness, burst pressure, and permeability. The preferred values for these characteristics can be obtained by varying the composition of the hydrogel. In the case of 3D-printed blood vessels, permeability sufficient to allow perfusion of red blood cells without leakage is preferred, a burst pressure of at lest 500 mmHg is preferred, and a toughness of at least 200 J m⁻² is preferred.

In some embodiments, the 3D-printed biomaterial comprises a first layer comprising the double-networked hydrogel, and a second layer comprising polyurethane mixed with a hydrogel component (e.g., gelatin or GelMA). In some embodiments, the polyurethane can be hydrolysis resistant polyurethane or thermoplastic polyurethane. For example, in some embodiments, comprising about 10-30% polyurethane and about 0-10% GelMA by weight. In further embodiments, the first layer comprises about 2% alginate by weight and about 10% gelatin by weight, while the second layer comprises about 20% polyurethane by weight and about 5% GelMA by weight. The first layer can provide a soft layer that is more cell-compatible, while the second layer is a more structurally strong layer. 3D-printed biomaterials comprising polyurethane have shown improved stability during storage of biomaterials such as blood vessels that include this material.

In some embodiments, the double-networked hydrogel comprises a bi-continuous emulsion. A bi-continuous emulsion is one in which two different polymer phases are present, to provide a copolymer biomolecule comprising alginate and gelatin or GelMA, present as macromolecules, and a template polymer such as poly(vinyl) alcohol or poly(ethylene oxide) that provides a medium (i.e., emulsion phase) for the copolymer. The copolymer can form a spectrum of continuous copolymer concentrations within the template polymer, resulting in a material having relatively lower concentration of the copolymer on one side (e.g., the top) and a relatively higher concentration of the copolymer on the other side (e.g., the bottom), with the concentrations gradually varying between the two sides. This can provide a biomaterial in which one side of the biomaterial is structurally strong, while the other side is softer and provides support for cell growth.

One of the polymers included in the hydrogel is gelatin methacryloyl (GelMA), also known as gelatine methacrylate or gelatin methacrylamide, is typically prepared by reaction of gelatin with methacrylic anhydride. Sun et al., Polymers (Basel)., 10(11): 1290 (2018). A variety of different concentrations of GelMA can be used. In some embodiments, the GelMA has a concentration ranging from 1% to 3% by weight, 2% to 4% by weight, 3% to 5% by weight, 1% to 5% by weight, 2.5% to 7.5% by weight, 5% to 10% by weight, 7.5% to 15% by weight, 10% to 15% by weight, 10% to 20% by weight, 15% to 20% by weight, or from 15% to 25% by weight.

The other polymer included in the hydrogel is alginate. Alginate is a natural anionic polymer typically obtained from brown seaweed consisting of linear copolymers of β-(1-4) linked d-mannuronic acid (M) and β-(1-4)-linked 1-guluronic acid (G) units. In some embodiments, the alginate has a concentration ranging from 0.1% to 1.5% alginate, from 0.1% to 3% alginate, from 0.5% to 2.5% alginate, from 1% to 3% alginate, or from 1% to 5% alginate, all by weight. In some embodiments, the alginate has a molecular weight from about 100 kDa to about 200 kDa, from about 200 kDa to about 300 kDa, or about 100 kDa. In some embodiments, the alginate has an M to G ratio (M/G) from about 0.3 to about 0.7, from about 0.7 to about 1.0, or from about 1.0 to about 1.3.

The 3D-printed biomaterial can be provided in a variety of different shapes. In particular, the biomaterial can be provided in shapes useful for medical applications. For example, in some embodiments, the biomaterial can be provided in the shape of an organ or tissue, such as circulatory organs. In some embodiments, the biomaterial comprises a hollow tube. A hollow tube can be used as a blood vessel, such as an artery or vein. The hollow tube comprises a cylinder having a hollow interior, an inner side facing the hollow interior, and an outer size facing the exterior of the cylinder. Blood vessels can have a variety of sizes, depending on the type of blood vessel, with a normal human aorta having a diameter of about 2 cm, while capillaries can have a diameter from about 2 to 12 μm. Accordingly, the blood vessels can have a diameter from about 2 μm to 2 cm, with non-capillary blood vessels having a diameter ranging from about 1 mm to about 2 cm.

In some embodiments, the cylinder forming the hollow tube can include multiple layers. When intended for use as a blood vessel, it can be desirable to include one or more layers of cells in the blood vessel, to provide the blood vessel with bioactivity and further biocompatibility. In some embodiments, the hollow tube comprises a middle layer of double-networked hydrogel, an outer layer of cells, and an inner layer of cells. In a further embodiment, the hollow tube comprises a middle layer comprising the double-networked hydrogel, an inner layer comprising endothelial cells, and an outer layer comprising smooth muscle cells. The middle layer of the blood vessel can have a thickness ranging from about 1 μm to about 2 mm, depending on the diameter of the blood vessel, while in some embodiments the middle layer has a thickness from about 5 μm to about 1 mm, while in further embodiments the middle layer has a thickness from 50 μm to 500 μm.

3D-printed biomaterials made to resemble blood vessels can exhibit vasoactivity. Vasoactivity refers to the ability of blood vessels to constrict or dilate (vasoconstriction and vasodilation, respectively) upon exposure to vasoactive agents, such as angiotensin, bradykinin, histamine, nitric oxide, and vasoactive intestinal peptide. While not intending to be bound by theory, incorporation of cells which respond to vasoactive agents appears likely to be behind the ability of 3D-printed biomaterials to exhibit vasoactivity.

In some embodiments, the hollow tube is an artery. An artery is a blood vessel that takes blood away from the heart to other parts of the body. More specifically, the hollow tube is a 3D-printed biomaterial made to represent an artery in terms of its size, shape, and characteristics. Arteries include the aorta, systemic arteries, arterioles, and capillaries. Arteries are designed to handle the higher pressure resulting from contractions of the heart. Natural arteries are surrounded by smooth muscle which includes extensive elastic and inelastic connective tissue. An artery includes three layers; the tunica externa (the outer layer), the tunica media (the middle layer), and the tunica intima (the innermost layer). In some embodiments, the 3D-printed biomaterial middle layer comprises an upper middle layer comprising 1% to 3% alginate by weight and 2% to 4% GelMA by weight, and a lower middle layer comprising 0.5% to 2% alginate by weight and 10% to 20% GelMA by weight. The upper middle layer has properties that encourage cell growth, while the lower middle layer has properties that provide mechanical strength.

In some embodiments, the hollow tube is a vein. A vein is a blood vessel that carries blood (typically deoxygenated blood) towards the heart. More specifically, the hollow tube is a 3D-printed biomaterial bade to represent a vein in terms of its size, shape, and characteristics. Veins include large, medium, and small veins (a.k.a. venules). Veins typically have less smooth muscle and connective tissue, and wider internal diameters than arteries. Like arteries, veins also include three layers. the tunica externa (the outer layer), the tunica media (the middle layer), and the tunica intima (the innermost layer). In some embodiments, the 3D-printed biomaterial middle layer comprises a hydrogel comprising 0.5% to 2% alginate by weight and 10% to 20% gelatin by weight.

The 3D-printed biomaterials can have essentially any size and shape that can be obtained using a 3D-printer. In some embodiments, the 3D-printed biomaterial is shaped as an object intended for medical or pharmaceutical use, such as a tissue scaffold (e.g., an artificial transplant support). In some embodiments, the 3D biomaterial is a soft tissue construct (e.g., an artificial organ). In some cases, the 3D biomaterial can be personalized for a specific subject by basing the 3D object on an image obtained from magnetic resonance imaging, computed tomography, or ultrasound. A wide variety of tissue engineering applications for 3D-printed biomaterials comprising hydrogels are known to those skilled in the art. Advincula et al., MRS Commun., 11(5):539-553 (2021).

In some embodiments, the 3D-printed biomaterial is a tissue construct. An example of a 3D-printed biomaterial tissue construct is neo-cartilage. In some embodiments, the 3D-printed biomaterial can be a soft tissue construct. Soft tissues connect and support other tissues and surround the organs in the body. They include muscles (e.g., the heart), fat, blood vessels, nerves, tendons, and tissues that surround the bones and joints. Examples of 3D soft tissue constructs include skin, musculoskeletal tissue, cardiac tissue, heart valve, liver, and neuronal tissue. The cells included in the tissue construct are preferably the type of cells normally found in the particular type of tissue, or precursor cells (e.g., stem cells) that will result in that particular type of tissue. The cells may be substantially uniformly distributed throughout the polymer, or they may be suspended within a part of the polymer.

Viable cells that can be included in a 3D-printed object include prokaryotic and eukaryotic cells. Non-limiting examples of eukaryotic cells include mammalian cells (e.g., stem cells, progenitor cells and differentiated cells). Stem cells have the ability to replicate through numerous population doublings (e.g., at least 60-80), in some cases essentially indefinitely, and also have the ability to differentiate into multiple cell types (e.g., pluripotent or multipotent). Other viable cells include immortalized cells that do not undergo normal replicative senescence, and can proliferate essentially indefinitely. Other living cells include embryonic stem cells, amniotic fluid stem cells, cartilage cells, bone cells, muscle cells, skin cells, pancreatic cells, kidney cells, nerve cells, liver cells, and the like. Viable cells are living cells.

Standard cell culture techniques are typically used when handling the cells for the 3D-printed biomaterial. For example, a portion of or the entire printed article can be placed under standard cell culture conditions (e.g., temperature, pressure, nutrient concentrations, etc.) in order for the cells to remain viable. The 3D-printed biomaterial can comprise from about 1×10¹ to about 1×10⁹ viable cells, or from about 1×10² to about 1×10⁸ viable cells, or from about 1×10³ to about 1×10⁷ viable cells, or from about 1×10⁴ to about 1×10⁷ viable cells, or from about 1×10⁵ to about 1×10⁷ viable cells (all being cells per milliliter).

The 3D-printed biomaterial can also include one or more additives. Non-limiting exemplary additives for the biomaterial include diluent synthetic polymers (e.g., polyethylene glycol, polypropylene glycol, poly(vinyl alcohol), poly(methacrylic acid)), drugs (e.g., antibiotics such as penicillin and streptomycin), cell nutrients (e.g., proteins, peptides, amino acids, vitamins, carbohydrates (e.g., starches, celluloses, glycogen), and minerals (e.g., calcium, magnesium, iron), synthetic or naturally occurring nucleic acids, absorbers to limit light penetration, inhibitors (e.g., scavengers and quenchers), refractive index modifiers (e.g., iodixanol), and nanocomposite components such as graphene or silica. The bioink formulation can comprise one or more additives in an amount of 0 wt % to about 25 wt % of the composition, based on total weight of the composition.

### Methods of Making Three-Dimensional (3D)-Printed Biomaterial

A further aspect of the invention provides a method of making a 3D-printed biomaterial. The method includes extruding a hydrogel comprising gelatin or GelMA and alginate from a 3D-printer; converting the hydrogel to a double-networked hydrogel by a) cross-linking the alginate with a first cross-linking method; and b) cross-linking the gelatin or GelMA with a second cross-linking method. In some embodiments, the hydrogel extruded by the 3D-printer comprises cells.

The hydrogel prepared using the method can have any of the combinations of polymers described herein. For example, in some embodiments, the alginate comprises 0.5% to 2% by weight and the gelatin (e.g., GelMA) comprises 10% to 20% by weight of the hydrogel.

In some embodiments, the hydrogel comprises a stronger layer having a higher stiffness and a smaller mesh size, and a weaker layer having a lower stiffness and a larger mesh size. In further embodiments, the stronger layer has a Young's modulus from about 50 to about 1000 kPa. In yet further embodiments, the weaker layer comprising 1% to 3% alginate by weight and 2% to 4% GelMA by weight, and the stronger layer comprising 0.5% to 2% alginate by weight and 10% to 20% GelMA by weight.

In some embodiments, viscoelastic phase-separation of the hydrogel is used to form a bi-continuous emulsion. Strong dynamic asymmetry between two components of a fluid polymer mixture can lead to viscoelastic phase separation. See Tanaka H., Phys. Rev. Lett. 76, 787 (1996) and Tanaka H., Commun. Physics 5, 1-12 (2022). A viscoelastic phase separation occurs when alginate gelatin (or GelMA) copolymer macromolecules are emulsified in a template polymer such as PVA or PEO and a phase separation is then allowed to occur. Aqueous two-phase emulsions (ATPEs) can form by liquid-liquid phase-separation due to thermodynamic incompatibility between the two hydrophilic non-oppositely charged phases, leading to the formation of a bi-continuous emulsion.

The method includes the step converting the hydrogel to a double-networked hydrogel by cross-linking. More specifically, the method includes cross-linking the alginate with a first cross-linking method, and b) cross-linking the gelatin or GelMA with a second cross-linking method. Use of two different cross-linking methods results in a double-networked hydrogel in which the gelatin and the alginate are cross-linked to form a network including different types of chemical associations between the polymers. For example, the first cross-linking method for cross-linking alginate can use ionic cross-linking using a divalent ion source (e.g., CaCl₂). Likewise, the gelatin (e.g., GelMA) can be using a different cross-linking method, such photopolymerization by UV irradiation or chemical cross-linking of glutamine and lysine amino acids using transglutaminase (e.g., microbial transglutaminase). In some embodiments, the first cross-linking method comprises using calcium chloride and the second cross-linking method comprises using transglutaminase.

The method can be used to create 3D-printed biomaterials having a variety of different shapes. In some embodiments, the extruded hydrogel comprises a scaffold, while in other embodiments the extruded hydrogel comprises a tissue construct such as a blood vessel. Different methods of 3D-printing can be chosen that are better suited to providing the specific shape of interest. In some embodiments, the bioink is applied using a vat printing polymerization method, such as stereolithographic printing, digital light processing, or volumetric printing. Levato et al., Nature Reviews Methods Primers, 3, 47 (2023). In some embodiments, microfluidics-enhanced bioprinting can be used. See Chatinier et al., biomicrofluidics, 15, 041304 (2021). Microfluid systems contain channels on the micrometer-scale and can facilitate precise positioning of different materials.

In other embodiments, such as when a hollow tube is being prepared, coaxial extrusion 3D printing can be used. A coaxial system includes a printhead configuration consisting of two needles in a coaxial arrangement allow for two separate fluid flows before they come in contact at the point of extrusion. A coaxial printhead can extrude the bioink and the cross-linking agent from the nozzle simultaneously, directly cross-linking polymers at the tip of the printhead. For example, a coaxial printhead can be used to extrude a hydrogel comprising a stronger layer having a higher stiffness and a smaller mesh size, and a weaker layer having a lower stiffness and a larger mesh size. Where a hollow tube is being extruded, the stronger layer can be adjacent to an inner surface, while the weaker layer is adjacent to an outer surface of the hollow tube.

In some embodiments, one or more surfaces of a hollow tube of 3D-printed biomaterial can be seeded with cells. Alternately, or in addition, the hydrogel forming biomaterial that is 3D-printed can include cells. In some embodiments, the method further comprises seeding the outer surface of the hollow tube with smooth muscle cells and seeding the inner surface of the hollow tube with endothelial cells. Cell seeding refers to spreading cells to a surface, such as a surface of a biomaterial. For example, cells can be seeded to an exterior surface of a hollow tube formed of 3D printed biomaterial by blocking off the ends of the tube and then placing the incubating the hollow tube for a period of time in a channel or other cell culturing space containing the desired cells. Cells can be seeded to the interior surface by, for example, perfusing cell suspension into the channel or other cell culturing space including hollow tubes seeded with cells on the exterior, and allowing the cells to adhere to the unoccupied interior surface of a hollow tube over an incubation period. Cells will typically adhere to a surface of the biomaterial within 24 hours of incubation.

In some embodiments, any one or more steps of the 3D printing method can be performed at a temperature from about 1° C. to about 99° C., or from about 10° C. to about 75° C., or from about 20° C. to about 50° C., or from about 25° C. to about 37° C. In some embodiments, all steps of the 3D printing method can be performed at a substantially constant temperature (e.g., no temperature change is required). Preferably, the 3D printing method is carried out at a temperature where cross-linking will result in fairly rapid polymerization of the polymer precursors, while being harmless to any cells that are present.

### Methods of Using 3D-Printed Blood Vessels

Another aspect of the invention provides a method of treating a damaged or diseased blood vessel in a subject. The method includes attaching a 3D-printed blood vessel to the damaged or diseased blood vessel of the subject and connecting the 3D-printed blood vessel to blood flow within the damaged or diseased blood vessel so that blood flow bypasses the damaged or diseased blood vessel and flows through the 3D-printed blood vessel. This procedure is also known as vascular bypass surgery. The 3D-printed blood vessel can be any of the 3D-printed blood vessels described herein. For example, the 3D-printed blood vessel can be a hollow tube comprising a middle layer comprising a double-networked hydrogel comprising gelatin and alginate, an inner layer comprising endothelial cells, and an outer layer comprising smooth muscle cells. The 3D-printed blood vessel can be attached to the damaged or diseased blood vessel by methods known to those skilled in the art, such as gluing or suturing.

Blood vessels include veins and arteries, and the 3D-printed blood vessel should have characteristics corresponding that are substantially similar to the blood vessel being replaced; e.g., if a diseased or damaged artery is being replaced, the 3D-printed blood vessel should be a 3D-printed artery. In some embodiments, the damaged or diseased blood vessel is an artery, while in further embodiments the damaged or diseased blood vessel is a coronary artery. In other embodiments, the damaged or diseased blood vessel is a vein.

A blood vessel can be damaged or diseased as a result of a variety of different events and conditions. For example, a blood vessel can be physically damaged as a result of accident or injury. Blood vessel damage, also known as vascular trauma, can be mild, moderate, or severe. A blood vessel can also be subject to a wide variety of different diseases. Examples of blood vessel diseases include peripheral artery disease, carotid artery disease, pulmonary embolism, collagen vascular disease, and atherosclerosis. Accordingly, in some embodiments, the damaged or diseased blood vessel is an atherosclerotic blood vessel.

Another aspect of the invention provides a method of testing a vasoactive drug. The method includes contacting a 3D-printed blood vessel with an effective amount of a vasoactive drug, and observing the effect of the vasoactive drug on the 3D-printed blood vessel. The 3D-printed blood vessel can be any of the 3D-printed blood vessels described herein. For example, the 3D-printed blood vessel can be a hollow tube comprising a middle layer comprising a double-networked hydrogel comprising gelatin and alginate, an inner layer comprising endothelial cells, and an outer layer comprising smooth muscle cells. An effective amount of a drug is an amount sufficient to induce a vasoactive effect in the 3D-printed blood vessel. As described herein, the 3D-printed blood vessels can exhibit vasoconstriction or vasodilation in response to contact with vasoactive drugs. This provides a method for testing the effects of vasoactive drugs in artificial blood vessels. Note that the 3D-printed blood vessels can also be used to test the response of blood vessels to other types of drugs, such as the response of blood vessels infected with a virus to an antiviral agent.

Blood vessels include veins and arteries, as known to those skilled in the art and as described herein. In some embodiments, the 3D printed blood vessel is a vein and wherein the middle layer comprises 0.5% to 2% alginate by weight and 10% to 20% GelMA by weight. In other embodiments, the 3D-printed blood vessel is an artery and wherein the middle layer comprises an upper middle layer comprising 1% to 3% alginate by weight and 2% to 4% GelMA by weight, and a lower middle layer comprising 0.5% to 2% alginate by weight and 10% to 20% GelMA by weight.

The method includes contacting a 3D-printed blood vessel with an effective amount of a vasoactive drug. In some embodiments, the vasoactive drug is a vasoconstricting agent. Examples of vasoconstricting agents include α-adrenoceptor agonists, vasopressin analogs, epinephrine, norepinephrine, phenylephrine, dopamine, dobutamine, and serotonin 5-hydroxytryptamine agonists. In further embodiments, the vasoactive drug is a vasodilating agent. Examples of vasodilating agents include angiotensin-converting enzyme inhibitors, angiotensin receptor blockers, calcium channel blockers, and nitrates.

The method includes observing the effect of the vasoactive drug on the 3D-printed blood vessel. The effect of the vasoactive drugs on the diameter of the 3D-printed blood vessel can be observed using, for example, a microscope, with increases in the diameter indicating vasodilation and decreases in the diameter indicating vasoconstriction.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Cell-Instructive Hierarchical Bi-Continuous Tough Hydrogels

The blood vessels are responsible for transporting blood cells, nutrients, and oxygen to the tissues of the human body, and for taking carbon dioxide and other wastes away. In terms of anatomical structures and functions, there are three main types of blood vessels: arteries, veins, and capillaries. While a capillary consists of only a layer of endothelial cells (ECs) supported by the subendothelial basement membrane and connective tissue, both arteries and veins have three concentric layers, i.e., tunica intima, tunica media, and tunica externa. Tunica intima is the innermost and thinnest layer and is mainly made up of ECs, which play pivotal roles in regulating coagulation, conferring selective permeability, and participating in transendothelial migration of circulating cells (S. P. Herbert, et al., Nat Rev Mol Cell Biol 12, 551-564 (2011)). Tunica media, the middle layer, mainly consisting of smooth muscles, especially in the veins and smaller arteries, controls the vessel's caliber and withstands blood pressures. In the arteries, tunica media becomes the thickest layer among the three, but in the veins, it is obviously thinner. Tunica externa, principally composed of connective tissues, serves as the outer layer (W. D. Tucker, et al., in StatPearls, StatPearls Publishing Copyright © 2021)).

Cardiovascular diseases (CVDs), such as ischemic heart disease, cerebellar stroke, and venous thrombosis, remain the leading causes of mortality and disability of human. Total prevalent cases doubled from 271 million in 1990 to 523 million in 2019, and the number of deaths kept increasing from 12.1 million to 18.6 million in the past 30 years. Revascularization is commonly achieved by bypass surgeries based on the grafting of autologous veins (e.g., the saphenous veins) and arteries (e.g., the internal mammary arteries), and these expensive procedures are performed over 400,000 times annually in the United States alone. However, autologous grafts for the heart and extremities have several drawbacks; specifically, the long-term patency rates remain low (A. L. Hawkes, et al., Vasc Health Risk Manag 2, 477-484 (2006)), and donor site injuries caused by invasive harvesting remain morbid for patients (B. McNichols, et al., Cardiol Ther 10, 89-109 (2021). Graft failure is largely driven by intimal hyperplasia and atherosclerosis (A. Malinska, et al., Histochem Cell Biol 148, 417-424 (2017)). As an alternative to autologous grafts, synthetic grafts, first introduced in the 1950s, made from poly(ethylene terephthalate) and expanded polytetrafluoroethylene, have been widely used (A. Lejay, et al., J Cardiovasc Surg (Torino) 61, 538-543 (2020)). However, small caliber synthetic grafts suffer from high rates of failure due to thrombogenicity, accelerated intimal hyperplasia at the distal anastomosis, and infectious complications (H. H. Greco Song, et al., Cell Stem Cell 22, 608 (2018)). The patency rates of synthetic substitutes are even lower than those of autologous grafts, where only 50% of synthetic grafts would survive 2 years in the peripheral circulation (A. L. Hawkes, et al., Vasc Health Risk Manag 2, 477-484 (2006)).

To overcome the limitations of autologous and conventional synthetic vascular grafts, various tissue-engineering strategies, including three-dimensional (3D) bioprinting, have emerged as potential vascular tissue-engineering approaches for fabricating viable vascular conduits. 3D bioprinting allows the recreation of vascular structures by precisely positioning biomaterials, cells, and possibly biologic signaling molecules (such as growth factors) to mimic their anatomical characteristics and facilitate tissue regeneration (X. Cao, et al., Engineering 7, 832-844 (2021)). Numerous bioprinting techniques, including extrusion-, inkjet-, and laser-based bioprinting, have been rapidly developed and applied in bioprinting of vascularized tissue constructs and perfusable vascular structures (P. Sasmal, et al., Microphysiol Syst 2, (2018)). In particular, a variation of extrusion-based bioprinting method, termed microfluidic coaxial bioprinting, can simultaneously deliver the bioink and the crosslinking agents as separate flow streams through a concentric nozzle, which allow single-step generation of standalone, hollow vascular conduits (Y. S. Zhang, et al., Nature Reviews Methods Primers 1, 75 (2021)). A wide range of sizes of the resulting vascular conduits with varying performances is conveniently attainable through microfluidic coaxial bioprinting using different nozzle setups and bioink designs. For example, Hong et al. successfully adopted a quick-gelling bioink to bioprint perfusable vessels (S. Hong, et al., Biomaterials science 7, 4578-4587 (2019)). Andrique et al. developed functional vesseloids with vasoactivities and permeability (Andrique, et al., Science advances 5, eaau6562 (2019)). Gao et al. reported tunable vascular equivalents containing endothelium by cell-laden alginate and extracellular matrix-based bioink as in vitro models (G. Gao, et al., Materials 31, 2008878 (2021)). The inventors also reported the bioprinting of cell-laden hollow fibers facilitating the proliferation and maturation of vascular cells (W. Jia, et al., Biomaterials 106, 58-68 (2016)), which could be further expanded to the engineering of cannular tissues of multiple circumferential layers (Q. Pi, et al., Advanced materials (Deerfield Beach, Fla.) 30, e1706913 (2018)), offering an approach for biofabrication of vascular conduits with different anatomical features.

Nevertheless, in many cases, these bioprinted vascular conduits only partially recapitulated the structural and functional features of the native blood vessels. More importantly, these vascular conduits possessed significantly weaker mechanical strengths than their native counterparts, limiting their biological applications under a physiological environment. Some other reports focused on developing synthetic polymer-based hybrid hydrogels, including nanocomposite hydrogels and double network (DN) hydrogels that convey high strengths and toughness (Q. Liang, et al., Advanced Functional Materials 30, 2001485 (2020)), which laid foundation for bioprinting of mechanical robust tubular structures. Although these synthetic polymer-based hydrogels are mechanically strong and cytocompatible, they may not support the spreading and proliferation of the embedded cells, limiting their desired biofunctions. Therefore, it remains a challenge to bioprint structurally similar, mechanically, and functionally relevant vascular conduits, particularly those serving as small-diameter vascular grafts.

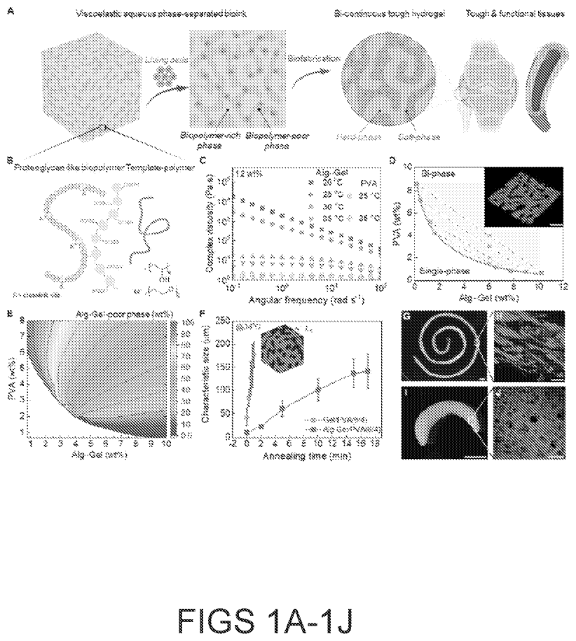

Here we report the design of microstructured bi-continuous tough hydrogel systems to tackle the long-lasting mechanical robustness-cellular function conflict, in addition to providing excellent processability for biofabrication of physiologically relevant tissues (FIG. 1A). In our approach, we combined aqueous viscoelastic phase-separation (AVPS) and DN hydrogel design to construct printable bi-continuous DN (BC-DN) tough hydrogels that exhibited outstanding mechanical properties and excellent bioactivities. The use of biopolymers and an immiscible template-polymer with asymmetric dynamics enabled the formation of bi-continuous emulsions with high phase stability and tunable self-assembly microstructures via AVPS. After bioorthogonal crosslinking, the biopolymer-rich phase-transformed into a load-bearing hard-phase to achieve high toughness competing with soft tissues. Meanwhile, the biopolymer-poor phase formed a high-diffusivity soft-phase to support favorable cell functions. We showcased the AVPS-emulsion bioinks for versatile biofabrication techniques, including injection-molding and bioprinting, to fabricate chondrogenic neo-cartilage and engineered vessels that exhibited physiologically relevant excellent mechanical properties (such as high suture-retention strength) and biological functions resembling the native vessels. Collectively, our work reveals how AVPS can be engineered to fabricate hierarchical tough hydrogels and for versatile biofabrication of physiologically relevant tissues with decoupled yet synergistic physical and biological functions.

### Engineered Bi-Continuous Emulsions Via AVPS

Conventional ATPEs exhibit low phase stability due to low interfacial energy and viscosity. Y. Chao, H. C. Shum, Chem Soc Rev 49, 114-142 (2020). The lack of suitable phase stability of ATPEs leads to poor control over microstructures and a limited time window for manufacturing. Ying et al., Adv. Mater. 30, 1805460 (2018). In AVPS-emulsions, the high apparent viscosity and prominent viscoelasticity facilitate unprecedented manipulation of the self-assembly microstructures. Using the proteoglycan-like copolymers and an immiscible hydrophilic polymer (template-polymer), we studied the emulsion compositions and micro-morphologies (FIG. 1B). The copolymers were synthesized by grafting alginate to gelatin (or gelatin methacryloyl, GelMA) macromolecules. In Alg-Gel (or Alg-GelMA) copolymers, gelatin (or GelMA) provides cell-adhesion and covalent crosslinking sites; meanwhile, alginate enables ionic crosslinking and water-retention by charged carboxylic acids. The resulting Alg-Gel macromolecules displayed a much higher viscosity (1-3 orders of magnitude) than template-polymers, including poly(vinylalcohol) (PVA-100 kDa) and poly(ethylene oxide) (PEO-300 kDa), of the same concentration at broad temperature range (FIG. 1C). This dynamic asymmetry between the biopolymer and the template-polymer forms AVPS emulsions. H. Tanaka, Communications Physics 5, 1-12 (2022).

Examining the phase-separation thermodynamics, we measured equilibrium phase ratios and compositions and plotted phase diagrams. After complete phase-separation, AVPS formed two layers: the bottom-layer biopolymer-rich phase and the top-layer biopolymer-poor phase. The mass ratio of the biopolymer-poor phase increased with the template-polymer concentrations for different biopolymers investigated, including gelatin, Alg-Gel, and Alg-GelMA. The modification of gelatin, including grafting alginate and methacrylate, also influenced the phase ratios. In the 2D phase diagrams, the bimodal curves separate the biphase region (above the curve) and the single-phase region (below the curve), and a tie line connects the two bimodal nodes associated with equilibrium concentrations (FIG. 1D). This allowed for precise control over the phase compositions and ratios by adjusting feeding concentrations. The phase diagrams can be manipulated for a given biopolymer by altering template-polymer types and molecular weights. The viscoelastic AVPS-emulsions formed bi-continuous microstructures featuring two interpenetrating continuous microphases in a wide range of feeding concentrations (FIG. 1D). The bi-continuous microstructures were reduplicated using leveled Gaussian-random-fields (Soyarslan et al., Acta Mater. 149, 326-340 (2018)), in which the average hard-phase sizes were fed as the modeling characteristic sizes. We used empirical models to fit the phase diagrams for numerical analysis (Lee et al., Fluid Phase Equilibria 508, 112441 (2020)). With this method, a color contour of biopolymer-poor 5 phase content as functions of Alg-Gel and PVA concentrations was plotted to guide the emulsion design (FIG. 1E). For example, with a constant 6 wt % Alg-Gel, elevated PVA contents from 1.5 to 5 wt % produced increasing biopolymer-poor phase content from 11 to 68 wt %.

To probe the phase-separation kinetics, we investigated the phase morphology at the macro- and micro-scales during mild heating (34-37° C.) (FIG. 1F). It is observed the domain sizes of AVPS (e.g., Alg-Gel/PVA) evolved much slower than conventional ATPEs (e.g., Gel/PVA). The bi-continuous microstructures of Alg-Gel/PVA emulsion showed increased phase sizes from 10 to 140 μm in 16 min at 34° C., which was captured by modeling. The enhanced stability of AVPS-emulsion provides a broad time window for (bio)fabrication.

### Bi-Continuous Tough Hydrogels Fabrication Using AVPS-Emulsions

We leveraged dual-crosslinking of AVPS-emulsions to form bi-continuous tough hydrogels. Alg-Gel (or Alg-GelMA) hydrogel precursors can proceed with rapid ionic crosslinking of alginate by metal ions (i.e., calcium chloride, CaCl₂) and enzymatic crosslinking of gelatin by TG (D. Wang et al., Sci. Adv. 8, eabg6900 (2022)), or photocrosslinking of GelMA by photoinitiator (M. Wang et al., Nat. Commun. 13, 3317 (2022)). The bioorthogonal crosslinking and tunable rheological properties of AVPS-emulsions were applied to various biofabrication techniques, including injectable molding and (bio)printing, to fabricate BC-DN hydrogels with multiscale structures. Using AVPS-emulsion (bio)inks, we achieved microfluidic coaxial (bio)printing of BC-DN hydrogel conduits with longitudinally aligned microstructure (FIG. 1G). We also realized direct extrusion (bio)printing of layered structures, including honeycombs and lattices, using viscoelastic AVPS-emulsions. Additionally, photocurable emulsions that exhibited enhanced transparency upon heating allowed for volumetric (bio)printing of microphase-separated 3D constructs, including meniscus, screw, bone, ear, and cube with channel (FIG. 1H).

### Mechanical Performance and Physiological Stability

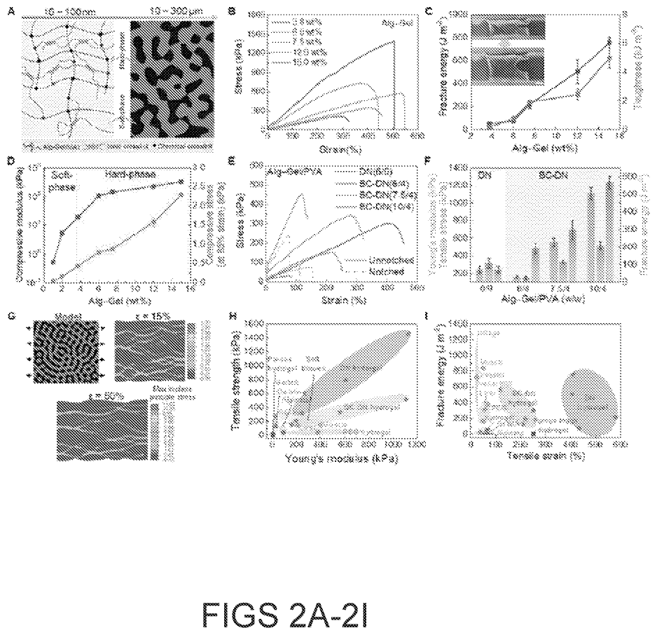

The BC-DN hydrogels showed hierarchal structures, including nanoscale networks and microphases (FIG. 2A). We hypothesized the interconnected energy-dissipative soft/hard hydrogels could enable excellent mechanical properties. To test this hypothesis, the tensile properties of alginate/gelatin-based DN hydrogels with varying compositions and concentrations were first measured. DN hydrogels with the alginate/gelatin mass ratio of 1/5 (w/w) showed the maximum fracture toughness; thus, the same ratio was used for copolymer synthesis. The Alg-Gel DN hydrogels displayed concatenation-dependent excellent mechanical properties (FIG. 2B). High Alg-Gel contents (15 wt %) formed hard DN hydrogels showing outstanding toughness (fracture energy, Γ: 754.8 J m⁻² toughness: 5.0 MJ m⁻³) (FIG. 2C). Reducing the biopolymer contents produced soft DN hydrogels showing low moduli (i.e., 0.3 kPa for 1 wt % Alg-Gel) (FIG. 2D). Consequently, BC-DN hydrogels with strong hard-phase and deformable soft-phase exhibited excellent tensile and compression properties (FIG. 2E). BC-DN hydrogels using 4 wt % PVA (191.0±29.1 J m⁻²) was 2.7 times as large as the DN hydrogel (71.0±19.0 J m⁻²) at the same biopolymer concentration. Because phase-separation enriched biopolymer of hard-phase and toughened BC-DN hydrogels. Further rise in biopolymer feeding concentrations, F of BC-DN hydrogels (Alg-Gel/PVA (7.5/4), Alg-Gel/PVA (10/4)) increased to 298.3±53.1 5 and 566.7±36.8 J m⁻², respectively (FIG. 2F).

We also evaluated the physiological stability and biodegradation profiles of the tough hydrogels. Due to ion-exchange of the alginate network, the stiffness and F of DN and BC-DN hydrogels declined after 2 hours of incubation in DPBS at 37° C. However, BC-DN hydrogels showed suitable stability in culture media, as indicated by highly preserved F (>70%) after 24 h in smooth muscle cell (SMC) culture medium. To validate the critical role of ion exchange on in vitro material degradation, we performed degradation tests in both DI water and DPBS at 37° C. In the case of DI water, BC-DN hydrogels shrank (around 30%) in 4 hours and remained stable for up to 2 months without apparent degradation. DN hydrogels of the same concentration (6 wt %) shrank 40% first and gradually swelled (12 wt %) during the two-months soaking. In DPBS buffer, however, both DN and BC-DN swelled first (10-20 wt %) and then gradually degraded to a residual mass of 50 wt % after 20 days, followed by a catastrophic degradation. These data suggest suitable stability and necessary degradability of BC-DN hydrogels for mechanically robust tissue scaffolds.

To illustrate the bi-continuous tough hydrogel versatility, we prepared BC-DN hydrogels using different template-polymer of PEO and photocurable biopolymers of Alg-GelMA. Remarkably, Alg-GelMA/PVA hydrogels showed an enhancement of 450% in F (32.0 J m⁻² to 177.0 J m⁻²) by increasing PVA content from 0 to 4 wt %. The toughening mechanism of BC-DN mainly lies in the stress-homogenization by the interconnected load-bearing hard-phase, as evidenced by finite-element analysis results (FIG. 2G). Together, BC-DN hydrogels displayed outstanding mechanical properties (Young's modulus<1.10 MPa, tensile strength<515 kPa, fracture strain<250%, and fracture toughness<566.7 J m⁻²) at low biopolymer feeding concentrations (<10 wt %). As a benchmark, these properties exceed most existing natural and synthetic biomaterial hydrogels, including gelatin, GelMA alginate, poly(ethylene glycol)-diacrylate, and their hybrids (Yang et al., Sci. Rep. 8, 1616 (2018); Drury et al., Biomaterials 25, 3187-3199 (2004); Nguyen et al., Biomaterials 33, 6682-6690 (2012)), as well as some soft tissues such as kidney, vessel, vocal fold, liver, muscle, and neo-cartilage.

### Cellular Activities and Generality

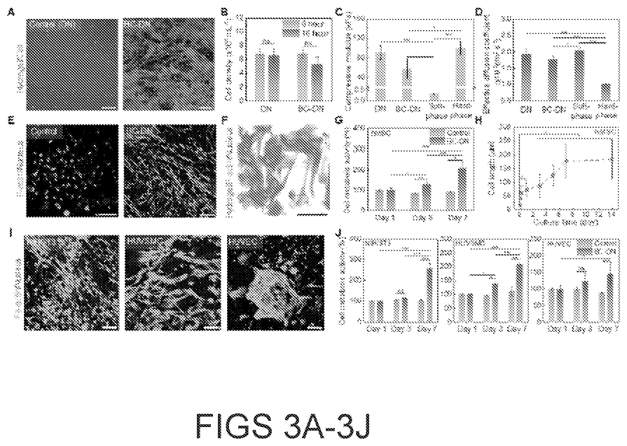

Envisioning that interconnected proteoglycan-like soft/hard hydrogels could mimic the structural and chemical features of ECM proteins, we examined how BC-DN hydrogels could accommodate favorable behaviors of residing cells. To understand cell-distribution and cell-matrix interactions, we exploited red-fluorescent emulsions to embed cell-tracker-labeled NTH/3T3 fibroblasts. The mixing conditions and thermal-annealing time before crosslinking controlled the cell distribution and self-assembly microstructures in BC-DN hydrogels. We premixed the cells in the biopolymer poor phase to selectively partition most cells in the soft-phase. Thermal annealing coarsened the phase size and drove cell attachment to the phase interface. Extended heating time (>5 min at 37° C.) resulted in large phase sizes (>200 μm) and partially diminished mechanical properties. Using controlled processing parameters, we obtained cell-laden BC-DN hydrogels consisting of a cell-rich soft-phase and a cell-poor hard-phase at the microscale (100-200 μm) (FIG. 3A). The cell density in cell-laden BC-DN hydrogels showed no significant difference with the emulsion bioinks (FIG. 3B). To understand the critical role of soft-phase in retaining high cell-density, we prepared porous-DN (P-DN) hydrogels by selectively leaching out the polymers in the biopolymer-poor phase before its gelation. With the rapid cell release, a significantly lower cell-preservation ratio (41%) was obtained in P-DN hydrogels. Therefore, the soft- and hard-phases synergistically contributed unique physiochemical properties of BC-DN hydrogels.

We hypothesized that the cell-benign microenvironments provided by the hard/soft phase in BC-DN hydrogel could enable good cellular functions. To test this hypothesis, we first measured the physicochemical properties of DN and BC-DN hydrogel and associated soft- and hard-phase hydrogels. Both DN hydrogel and BC-DN hydrogel's hard-phase exhibited high stiffnesses (compressive modulus: 90.5 and 99.5 kPa) (FIG. 3C) and low effective diffusivity coefficients (Deff: 1.8×10⁻⁶ and 1.0×10⁻⁶ cm s⁻²) (FIG. 3D), respectively. In contrast, BC-DN hydrogel's soft-phase (0.7 wt % Alg-Gel) showed an extremely low stiffness (0.3 kPa) and a high Deff (2.0×10⁻⁶ cm s⁻²). This revealed distinct network mesh sizes of the hard-phase (20 nm) and the soft-phase (359 nm). Therefore, soft-phase enabled fast nutrient/oxygen diffusion and waste removal, promising suitable cellular activities in BC-DN hydrogels.

Evaluating the cellular activities in tough hydrogels, we employed human mesenchymal stem cells (hMSCs) as model cells. With live/dead assay, it was observed an increased significantly higher viability (92%) in BC-DN hydrogels compared with that in DN hydrogels (78%) on day 7. Unlike negligible cell-spreading in DN hydrogels, BC-DN hydrogels enabled apparent cell elongation, as shown by F-actin staining results (FIG. 3E). Surprisingly, BC-DN hydrogels supported rapid hMSC spreading as early as 6 hours of embedding. Using red-fluorescent BC-DN hydrogels, we identified the hMSCs attachment to the hard/soft phase interfaces and spreading across the continuous soft-phase (FIG. 3F). The heterogeneous BC-DN hydrogels supported favorable cellular functions for up to 14 days evaluated without microstructure distortion. As expected, hMSC metabolic activities were enhanced by 100% in BC-DN hydrogels compared to the minor reduction in DN hydrogels on day 7 (FIG. 3G). Consequently, cell length increased from 15.6±3.1 μm to 183.7±69.8 μm during the 14-day culture (FIG. 3H). Probing further, we measured the compressive properties of hMSC-laden BC-DN hydrogels, indicating a slight rise in stiffness on day 14, likely due to ECM-secretion by the residing cells. After soaking in 3 w/v % CaCl₂, significantly boosted mechanical properties were obtained for all cell-laden BC-DN hydrogels.

To examine the versatility of BC-DN hydrogels in supporting good cellular activities, we then used different cell types and hydrogel materials. Noticeably better cellular functions in BC-DN hydrogels over DN hydrogels were observed using other cell types, including NIH/3T3 mouse fibroblasts, human umbilical vein smooth muscle cells (HUVSMCs), and human umbilical vein endothelial cells (HUVECs) (FIGS. 31 and J). Using much tougher BC-DN hydrogels with higher biopolymer concentrations, suitable cellular activities were indicated by viability above 85% and cell elongation after 7 days. Besides, photocurable BC-DN hydrogels supported high viability (92%) and prominent cell proliferation. Therefore, the bioactive BC-DN hydrogels with good cell adhesions, suitable viscoelasticity, and nutrition transport are favorable candidates as tissue scaffolds.

### Biofabrication of Chondrocyte-Laden Tough Hydrogel as Neo-Cartilage

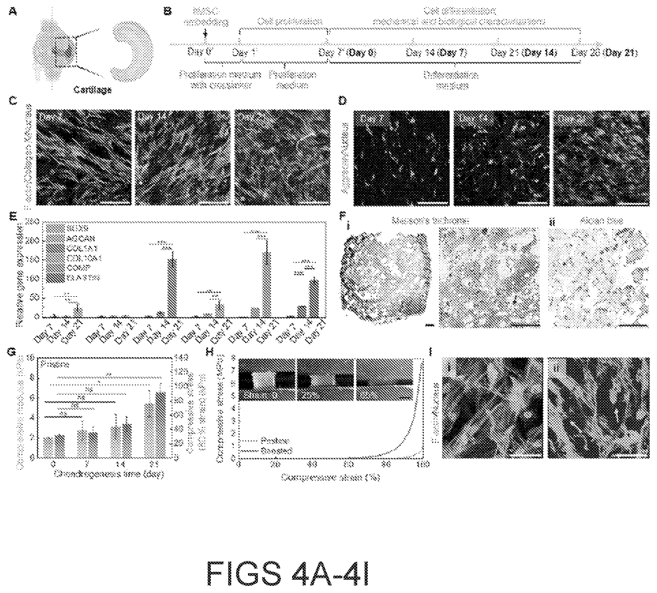

Because the tough microstructured hydrogels can be used as tissue scaffolds to accommodate good cellular behaviors, it is possible to biofabricate mechanical-robust complex engineered tissues that are not attainable via current biomaterials. To demonstrate how the physiological properties regulated tissue formation, we studied the chondrogenesis behaviors of hMSC-laden BC-DN and DN (as control) hydrogels (FIG. 4A). First, the hMSC-laden hydrogel precursors were injected into molds to form cell-laden tough hydrogels, followed by 7-day proliferation and 21-day chondrogenic differentiation (FIG. 4B). Co-staining of F-actin and collagen type X results suggested that hMSCs formed abundant actin filaments and collagen within the elongated cells in the BC-DN hydrogels by chondrogenic differentiation (FIG. 4C). The increased deposition of aggrecan also evidenced progressive chondrogenesis (FIG. 4D). By contrast, DN hydrogels restricted cell spreading and showed suppressed differentiation. The prominent expressions of specific chondrogenic genes in hMSC-laden BC-DN hydrogels confirmed the chondrogenic differentiation (FIG. 4E). Compared with DN hydrogels, BC-DN hydrogels facilitated highly enhanced expressions of several chondrogenic genes (such as COMP, COL10A1, and ELASTIN). Using histological examinations, the collagen deposition by Masson's trichrome and aggrecan deposition by Alcian blue staining in BC-DN hydrogels were visualized, revealing the prominent cellular functions. Because the two-phase microstructures allowed good cell-phase interface interactions and cell-cell communication. Conversely, lack of cell spreading and contact in DN hydrogels yielded poor biological functions (FIG. 4F).

The engineered neo-cartilage exhibited improved mechanical properties during chondrogenesis. The neo-cartilages showed an enhancement of 1.6-fold in compressive modulus and 1.9-fold in compressive stress (at 80% strain) after 3-week chondrogenesis (FIG. 4G). This observation highlighted the mechanical-support role of ECM (such as collagen fibrils) secreted by the residing cells. The aligned collagen microfiber could deform and rupture for effective energy dissipation on top of the tough matrix (FIG. 4I). Remarkably, the chondrogenic neo-cartilage displayed high compression strengths of 6-8 MPa after boosting the mechanical properties (FIG. 4f). Our results of enhanced mechanical properties of the engineered neo-cartilage are consistent with previous work. Nimeskern et al., Tissue Engineering Part B: Reviews 20, 17-27 (2014).

### Biofabrication of Physiologically Relevant Blood Vessels

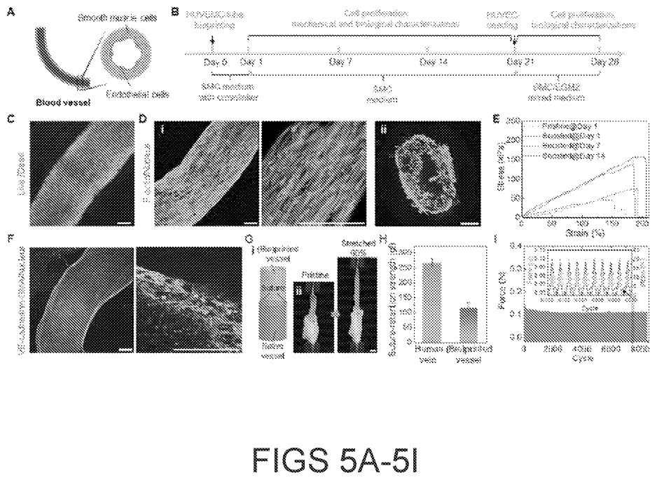

We also showcased the biofabrication of strong and functional vascular conduits based on BC-DN hydrogels (FIG. 5A). To do this, an HUVSMC-laden emulsion bioink was first coaxially bioprinted for 3-weeks vitro culture (FIG. 5B). The coaxially bioprinted cell tubes of various sizes (1.5-4.3 mm of out-diameter) exhibited high viability above 85% (day 1). Increased viability above 93% and apparent cell spreading were observed after 7 days post-printing (FIG. 4C). Additionally, F-actin staining results further illustrated prominent HUVSMC spreading and

Meanwhile, bioprinted HUVSMC-tubes exhibited outstanding mechanical properties. The cell-tubes showed Young's moduli of 20.3±2.3 kPa and 179.6±9.6 kPa, and 5 break strengths of 29.5±7.5 kPa and 163.7±2.4 kPa before and after boosting in CaCl₂ on day 1, respectively (FIG. 4E). Noted the outstanding cellular functions of cell-tubes were not impacted by boosting the mechanical properties by CaCl₂) (3 w/v %) soaking during culture (FIG. 4D). However, the mechanical booting step greatly impacted the mechanical responses and fracture behaviors of HUVSMC-tubes. After the tensile fracture, the pristine cell-tubes with low stiffness ruptured into cell clusters featuring smooth propagating cracks. In contrast, the boosted cell tubes maintained highly aligned cellular morphology supported by the strong hydrogel matrix. Because the strong and tough matrix governed the mechanical properties of the cell-tubes, as suggested by comparable mechanical properties of freshly (bio)printed acellular and cellular tubes. The mechanical properties of the cell-tubes gradually decayed with extended culture periods, due to matrix degradation enhanced by cell remodeling. Schulz et al., Proc. Natl. Acad. Sci. U.S.A 112, E3757-E3764 (2015). Nevertheless, cell tubes still showed a tensile strength of 78 kPa on day 14 (FIG. 4E). On day 21, HUVECs were post-seeded in the lumen for an additional 7-day co-culture. Using immunostaining, we observed the expression of endothelium-specific marker (VE-cadherin) for HUVECs in the lumen and α-smooth muscle actin (α-SMA) by HUVSMCs in the sheath layer (FIG. 4F). As indicated, biologically relevant vessels were formed.

The mechanically and biologically relevant engineered vessels promise great translation potential. We demonstrated proof-of-concept hand-sutured anastomosis of (bio)printed tough conduits and native human veins. The anastomosed conduits enabled perfusion and maintained structural integrity up to 60% of stretching strain (FIG. 4G). High suture-retention strength of 115±15 gram-force (gf) was achieved for acellular tubes (FIG. 4H), highlighting its translation potentials. L. E. Niklason, J. H. Lawson, Science 370, 5 eaaw8682 (2020). Moreover, the anastomosed conduit exhibited good resilience and outstanding mechanical durability upon physiological deformation (20%) over 8,000 times (FIG. 4I). Therefore, compared with most existing tissue-engineered vessels, our BC-DN hydrogels-based engineered vessels showed prominent advantages in synergistic good mechanical properties, biological functions, and processability. Gao et al., Applied Physics Reviews 6, 041402 (2019); Andrique et al., Sci. Adv. 5, eaau6562 (2019).

### DISCUSSION

Microstructured heterogeneous tough hydrogels by AVPS and DN design simultaneously achieved good processability, high toughness/strength, and excellent cellular functions, which were applied for the versatile biofabrication of physiologically relevant engineered tissues, including cartilage and blood vessels. Although the toughness of our heterogeneous tough hydrogel-based engineered tissues exceeded most existing engineered tissues, they still cannot compete in vivo-matured tissues in physiological stability and biological functions. Accordingly, future work is needed to realize de novo fabrication of physiological-stable and fully functional tissues. This includes developing novel AVPS-based tough biomaterials, assembling/alignment of multiple cells, and dynamic stimulation-mediated post-culture. There is also a need to engineer the matrix degradation profile to match the cell remodeling rate for different tissues. However, the present work provides the first proof-of-concept biomaterial design method of accommodating biofabrication toward achieving excellent mechanical properties while accelerating functional tissue formation.

In addition to their important use in biofabrication and tissue engineering, the hierarchal tough hydrogels formulated by viscoelastic liquid-liquid phase-separation strategies can be a robust and versatile approach to large-scale fabrication of multiscale hierarchal soft materials with decoupled yet synergistic physicochemical properties and multifunctions. The AVPS-based heterogeneous tough hydrogel concept can be extended to multi-material and other tough hydrogel designs to achieve broad applications. This includes the application in sequentially controlled drug delivery systems with decoupled mechanical properties and diffusivity and high-performance hydrogels with good mechanical properties and conductivity. Additionally, the micro- and nano-scale self-assembling of AVPS can greatly empower advanced manufacturing with multiscale structure-, composition-, and property-manipulations. Considering the versatile aqueous phase-separation phenomena among natural and synthetic polymer systems, this AVPS-based heterogeneous tough material concept is an important design paradigm for next-generation biomaterials and future advanced (bio)manufacturing, in which decoupled functional properties and multiscale structures are needed.

### Materials and Methods

Gelatin from porcine skin (Type-A, 50-100 kDa, 300 bloom, G2500), methacrylic anhydride, sodium alginate from brown algae (Mn~200 kDa, 71238), PVA (Mn=31-50 kDa (363138), 89-98 kDa (341584)), PEO (Mn=40 kDa (95904), 300 kDa (182001)), N,N-dimethylformamide (DMF), sodium hydroxide (NaOH), dimethyl sulfoxide (DMSO), 2-(N-morpholino)ethanesulfonic acid solution (MES, M1317), N-hydroxysuccinimide (NHS, 130672), 1-ethyl-3-(3-dimethylaminopropyl)carbodiimide (EDC, 03450), CaCl₂) (C5670), bovine serum albumin (BSA, A7030), triton X-100 (1003287133), L-proline (P0380), Insulin-Transferrin-Sodium Selenite (ITS) Supplement (11074547001), L-ascorbic acid (A92902), dexamethasone (D4902), 10% formalin solution (HT501320), and collagenase type IV (C4-28) were obtained from Sigma-Aldrich. Polydimethylsiloxane (PDMS) (Sylgard Silicone Elastomer 184) was ordered from Dow chemical. Dialysis membrane tubing (MWCO: 12-14 kDa) was purchased from Spectrum Laboratories. TG was supplied by Ajinomoto North America. Tris(2,2-bipyridyl)dichlororuthenium(II) hexahydrate (Ru) and sodium persulfate (SPS) were obtained from Advanced BioMatrix. Dulbecco's phosphate-buffered saline (DPBS), fetal bovine serum (FBS), Dulbecco's modified Eagle medium (DMEM), trypsin-EDTA, and antibiotic-antimycotic solution (Anti-Anti, 100×) were supplied from Life Technologies. Endothelial Cell Growth Medium-2 BulletKit (EGM-2) with SingleQuots Supplements, MSC Growth Medium BulletKit with SingleQuots Supplement Kit, and transforming growth factor beta-3 (TGF-β3) (PT-4124) were obtained from Lonza. The SMC Basal Medium 2 with Growth Medium 2 SupplementPack were obtained from PromoCell. CellTiter 96@AQueous Non-Radioactive Cell Proliferation Assay (4,5-dimethylthiazol-2-yl)-5-(3-carboxymethoxyphenyl)-2-(4-sulfophenyl)-2H-tetrazolium, MTS) (G3580) was obtained from Promega. 4′,6-diamidino-2-phenylindole (DAPI) (D1306), Live/Dead Viability/Cytotoxicity Kit (L3224), Invitrogen™ CellTracker™ Green CMFDA Dye (C7025), Alexa Fluor™ 488-phalloidin (A12379), and mouse anti-Aggrecan monoclonal antibody (MA3-16888) were purchased from Thermo Fisher. Mouse anti-collagen X antibody (ab49945), rabbit anti-VE-cadherin antibody (ab205336), mouse anti-α-SMA antibody (ab7817), and Alexa Fluor™ 594- or 488-conjugated goat anti-rabbit or goat anti-mouse secondary antibodies were purchased from Abcam. All chemicals were used as received.

### Biopolymer Copolymer Synthesis

### Alginate-Gelatin Copolymer (Alg-Gel)

Alg-Gel was synthesized via EDC/NHS coupling reaction between alginate and gelatin. Briefly, 200 mL of 2 w/v % sodium alginate (1×10⁻² mmol, 1 eq) (pH=5.5 adjusted by MES solution) was activated by EDC (2×10−2 mmol, 2 eq) and NHS (2×10⁻² mmol, 2 eq) at 30° C. for 30 min. 10 g of gelatin was dissolved in 200 mL of DPBS at 50° C. to obtain a 5 w/v % solution followed by cooling down to 30° C. Then, the NHS-activated alginate solution was slowly added to the gelatin solution with magnetic stirring (pH=6.5 by 1M NaOH), and the reaction was kept at 30° C. for 4 hours. The resulting solution was dialyzed against deionized (DI) water using dialysis membranes for 4 days at 37° C. The purified solutions were sterile-filtered (0.22 μm) (Millipore®, Sigma-Aldrich) and lyophilized to yield Alg-Gel copolymer (alginate/gelatin=1/5, w/w). The obtained foam was stored at −20° C. for later use.

### Alginate-GelMA Copolymer (Alg-GelMA)

A one-pot sequential methacryloyl reaction and EDC/NHS coupling reactions were exploited to synthesize Alg-GelMA. To 10 g of gelatin in 100 mL of DPBS (10 w/v %), 0.2 mL of methacrylic anhydride was slowly added, and the reaction was left at 50° C. for 2 hours. The solution was diluted with 100 mL of DPBS and cooled down to 30° C. To this solution, 200 mL of NHS-activated alginate (1 w/v %) was slowly added, and the reaction was maintained at the same temperature with continuous stirring for 12 hours. The obtained solution was dialyzed, sterile-filtered (0.22 μm), and freeze-dried to obtain the Alg-GelMA copolymer. The foam was stored at −20° C. for later use.

### Rhodamine-B-Conjugated Polymer Synthesis

Rhodamine-B-conjugated biopolymers were synthesized by EDC/NHS coupling between biopolymers and Rhodamine-B. Briefly, 0.192 g of rhodamine-B (0.4 mmol, 1 eq) was dissolved in 20 mL of DMF, and then 0.046 g of NHS (0.4 mmol, 1 eq) and 0.077 g of EDC (0.4 mmol, 1 eq) were added in sequence and then left to react under stirring at room temperature for 3 hours. 1.2 g of copolymer (Alg-Gel or Alg-GelMA) or 1.0 g of gelatin was dissolved in 20 mL of DPBS. Then 1 mL of NHS-activated rhodamine-B solution was added slowly into the above solution at 30° C., and the reaction was kept at 30° C. for 24 hours in the dark. The reaction solution was loaded in dialysis membranes and dialyzed in DI water at 37° C. until the waste liquid had no visible color through changing the DI water. The obtained solution was sterile-filtered (0.22 μm), freeze-dried, and stored at −20° C. for later use.

### Rheological Characterizations

Rheology measurements were conducted on a DHR-3 rheometer (TA Instruments) with a 40-mm diameter, 1° core plate geometry (steel Peltier plate). The plates were heated to 37° C. before loading the samples, and any excess samples were trimmed after lowering the gap height to 26 μm. The apparent viscosities as a function of shear-rate were measured from 0.1 to 100 s⁻¹ using a steady-state shear rate-sweep at 37° C. Steady-state temperature-sweeps were conducted at a shear rate of 1 s⁻¹ over the range of 15 to 37° C. Oscillatory temperature sweeps were performed from 15 to 37° C. with a shear strain of 1% and a frequency of 6.28 rad s⁻¹. Oscillatory frequency-sweeps were carried out from 0.1 to 100 rad s⁻¹ with a strain of 1% at various temperatures ranging from 15 to 40° C. (5° C. intervals). Oscillatory time-sweep was performed at a frequency of 6.28 rad s⁻¹ and a shear strain of 1% at 37° C.

### FTIR Characterizations

Chemical compositions of biopolymer powders or freeze-dried foams were characterized by a Fourier-transform infrared (FTIR) spectroscope (iS50 FTIR spectrometers, Thermo Fisher) in attenuated total reflectance mode. FTIR spectrum was recorded by averaging 32 scans of the signal at a resolution of 2 cm⁻¹ from 400 to 4,000 cm⁻¹.

### Biopolymer Solution and Emulsion Preparation and Characterizations

Preparation procedure: Aqueous biopolymer solutions and emulsions were prepared by mixing concentrated stock solutions. Briefly, 15 wt % or 12 wt % biopolymer stock solutions and 12 wt % template-polymer stock solution were first prepared by dissolving the lyophilized foam and polymer powder in DI water under heating. The solutions and emulsions with various mass concentrations were prepared by mixing the predetermined amount of biopolymer solution, template-polymer solution, and additional DT water using a spatula, followed by centrifugation to remove air-bubbles. The biopolymer solution or emulsion was denoted as biopolymer/template (x/y), where biopolymer is gelatin, Alg-Gel, or Alg-GelMA, the template is PVA or PEO, and x and y are the weight fractions of respective components. Unless otherwise noted, PVA (100 kDa) was used as the template-polymer.

Phase-separation visualization: Red-fluorescent emulsions was prepared by replacing 10 wt % biopolymers with relevant rhodamine-B-conjugated biopolymers. The macroscopic stability of red-fluorescent emulsions was evaluated by recording the morphology during the treatment in a 37° C. oven using a digital camera (Canon). The microscale morphology was captured using thin-film samples sandwiched between glass slides on fluorescence microscopes for ex-situ (ECLIPSE Ti, Nikon) or in-situ (Axio Observer, Zeiss) observation, as well as on a confocal fluorescence microscope (Olympus FV3000).

### Phase-Diagram Quantification

The aqueous emulsions of various concentrations were treated with heating at 70° C. for 20 min and centrifuging at 5,000 rpm for 20 min twice for complete phase-separation. After cooling to 4° C., the bottom phase (biopolymer-rich phase) in a physical gel and the liquid top phase (biopolymer-poor phase) were independently collected. The binodal curve was determined by calculating the concentration of each component in the top and bottom phases. The biopolymer-poor phase content (a) (the mass ratio of top-layer aliquot to the total system) and the mass of freeze-dried aliquots from both phases were obtained by analytical balance (Mettler Toledo) with a precision of ±0.0001 g. The above data was fed into mass balance and bimodal-model equations to determine the concentration of components in each phase.

### DN and BC-DN Hydrogel Preparations

The aqueous biopolymer solutions and emulsions were used as hydrogel-precursors to prepare the hydrogels. Gelatin/alginate DN hydrogels were prepared using 12.5 wt % gelatin and varying alginate contents (0.5 to 3 wt %). Alg-Gel DN hydrogels were prepared using Alg-Gel solutions of various concentrations ranging from 1 to 15 wt %. Alg-Gel-based BC-DN hydrogels were fabricated using emulsions of various biopolymer concentrations and template-polymer types and concentrations. The bubble-free hydrogel-precursors mixed at 37° C. were quickly cast and sandwiched between two acrylic plates with a 0.3-mm spacer. The samples were annealed at 37° C. for 2 min and then cooled at 4° C. for 5 min to form a physical-gel before transferring into a room-temperature crosslinker solution (3 w/v % CaCl₂ and 2 w/v % TG in DI water). After 20 min, the hydrogels were further cured in the presence of the crosslinker overnight at 37° C. To fabricate Alg-GelMA-based hydrogels, hydrogel-precursors with 0.5-mM/5.0-mM Ru/SPS as the photoinitiator were photocrosslinked between acrylic plates under a visible-light LED (20 W, Jobmate) for 60 s on each side. The soft films were further cured in 3 w/v % CaCl₂ overnight at 37° C. Additionally, cubic samples (4-mm side length) were prepared using PDMS molds. The crosslinked hydrogels were rinsed with DI water and used for further characterization.

### (Bio)Printing Procedure

Two different-sized blunt needles (SAI Infusion Technologies) were concentrically fixed as a core and sheath layer using a 2-part epoxy adhesive (Gorilla). The assembled nozzles were connected with syringes via Luer-lock adaptors (RSN Lab) and silicon tubes (Uxcell). Two independent syringe pumps (Braintree Scientific) controlled the flows of crosslinker (0.3 w/v % CaCl₂) in the core layer and emulsion (bio)inks in the sheath layer with a speed of 200 μL min⁻¹ and 100 μL min⁻¹, respectively. (Bio)inks extruded under gentle heating formed tubes in a bath containing 0.01 w/v % of CaCl₂. The resulting tubes were further cured in a crosslinker solution (3 w/v % CaCl₂ and 2 w/v % TG) overnight at 37° C.

Extrusion (bio)printing: The Alg-Gel/PVA (6/2) ink was loaded in a 10 mL syringe and centrifuged to remove the air bubbles before use. The print was conducted on an Allevi 2 bioprinter (3D Systems) using a 25G blunt needle at 28° C. The printed green parts were cured in the crosslinker solution (3 w/v % CaCl² and 2 w/v % TG) at 37° C.

Volumetric (bio)printing: Volumetric (bio)printing was conducted on a customized printer with a visible-light LED light engine (PRO4500, Wintech). Alg-GelMA/PVA (6/2) emulsion consisting of 0.5-mM/5.0-mM Ru/SPS was loaded in a 2-mL clear glass shell vial and centrifuged to remove the air bubbles. A projection algorithm was used to generate the patterned intensity-modulated images by custom MATLAB scripts (MATLAB 2022). The digital models were designed in SolidWorks (Dassault Systemes). The (bio)ink was rotated for green light illumination (525 nm) in approximately 60 s. The uncured ink was removed by gently rinsing with warm DI water, and the green part was treated with 3 w/v % CaCl₂.

### Mechanical Tests

### Tensile Test

All the mechanical properties were measured on a mechanical testing machine (100-N load-cell, Instron 3342). Rectangular samples (0.3-0.2 mm in thickness, 6-10 mm in width) were cut from hydrogel films for uniaxial tension tests. A gauge length of roughly 5-mm (Lₒ) and a stretch rate of 100% min⁻¹ were used in all tensile tests unless otherwise noted. The normal stress (σ) and strain (ε) are defined as the measured force divided by the initial cross-sectional area and the displacement divided by the initial gauge distance, respectively. Young's modulus was determined by a linearly fitted slope below 3% strain, and tensile stress/strain was obtained by averaging the ultimate stress/strain from stress-strain curves (n=3-5). Toughness was calculated by areal integration under stress-strain curves using Origin Pro 2018 (OriginLab).

### Fracture Energy Evaluation

To measure fracture energy, unnotched and notched samples with the same dimensions were tested using the same conditions. The notched samples with a 2-mm precut in the middle were stretched for crack propagation (n=2-3). The critical strain (ε_{c}) was determined from the strain at peak stress in notched samples. The fracture energy (I′) was calculated by the areal integration under the stress-strain curve for unnotched specimen until ε_{c} as

$$Γ = {L}_{0} {\int }_{0}^{{ε}_{c}} σ d ε .$$

Compression test: Cubic samples (approximately 4 mm in side-length) were tested on a mechanical testing machine (100-N load-cell, Instron 3342) using a compressing rate of 50% min⁻¹. Compressive modulus was calculated by linearly fitting stress-strain curves within 5-10% strain (n=2-3).

### Finite-Element Analysis

The uniaxial stretching of BC-DN hydrogels was simulated using an FEA software ABAQUS (v6.13, Dassault Systemes). The two-dimensional (2D) bi-continuous microstructures with a characteristic-size of 80 μm and soft-phase ratio of 43% were generated by MATLAB scripts based on 2D Gaussian-random-field. The exported phase boundary coordinates were used to create the 2D models (3.5×3.5 mm²) by running customized python scripts. The input parameters used were: hard-phase Young's modulus=430 kPa, soft-phase Young's modulus=1 kPa, Poisson's ratio of soft- and hard-phases=0.45. The maximum in-plane principal stress profiles were recorded during the stretch up to 50% strain.

### Diffusion Coefficient Measurement

The solute diffusivity in hydrogels was measured. Hydrogel precursors with 1 w/v % TG were half-filled in rectangular cuvettes (2×10×45 mm³) and cooled at 4° C. After adding crosslinker solution (3 w/v % CaClₕ and 2 w/v % TG), the curing was conducted in sealed cuvettes overnight at 37° C. The trapped hydrogels were rinsed with DI water. To measure the diffusion coefficient, 0.1 mg mL⁻¹ of rhodamine-B was quickly added to the top of the hydrogels in a cuvette, and fluorescence images were taken at different time intervals on a fluorescence microscope (Zeiss). The fluorescence intensities were normalized by 1 and 0 at the opening and end of the cuvette using ImageJ (v1.53s, National Institutes of Health), respectively. The intensity profiles below 0.6 were fitted by the one-dimensional diffusion equation

$$F ( x , t ) ∝ erfc ( \frac{x - a}{2 \sqrt{{D}_{eff} t}} )$$

to obtain the effective diffusion coefficient (D_{eff}, cm² s⁻¹), where erfc is the complementary error function, F is the normalized fluorescence intensity, x is the distance from the opening side (cm), a is a distance calibration constant (cm), and t is the contact time (s).

### Cell-Laden Hydrogel Preparations

Cell-laden hydrogels were prepared by blending living cells in emulsions for dual-crosslinking. The BC-DN hydrogels were made from Alg-Gel/PVA (6/4) emulsion unless otherwise noted. Cell-laden DN hydrogel (as a control) were fabricated using 6 wt % Alg-Gel. Hydrogel-precursors were sterilized by heating (70° C.) and cooling (4° C.) (10-min each) for 4 cycles. NIH/3T3 fibroblasts (passage 7-9, American Type Culture Collection, CRL-1658™) in DMEM supplemented with 10 v/v % FBS and 1 v/v % Anti-Anti, hMSCs (passage 4-6, Lonza) in MSC growth medium with supplements and 1 v/v % Anti-Anti, HUVSMCs (passage 5-8, ScienCell Research Laboratories) in SMC growth medium with supplements and 1 v/v % Anti-Anti, and HUVECs (passage 5-7, Angio-Proteomie) in EGM-2 medium with supplements and 1 v/v % Anti-Anti, were cultured in the incubator (Forma Scientific) with 5% CO₂ at 37° C. The respective culture media were changed every 2-3 days, and cells were passaged at 85% confluence. The cells were trypsinized using 0.05 v/v % trypsin-EDTA, centrifuged, and resuspended in FBS. Subsequently, cell suspensions were mixed with hydrogel-precursors to prepare cell-laden bioinks (5-10×10⁶ cells mL⁻¹). For emulsion bioinks, cells were first blended with the phase-separated top-phase and gently mixed with the bottom-phase in a warm water bath. The Alg-Gel/PVA(10/4) emulsion is very viscous, so cells were directly blended with the emulsions. The bioinks were injected into PDMS molds to cast films (1-mm-thick) and cubes (4 mm of side length). The samples were annealed at 37° C. for 2 min before curing with a filtered crosslinker (0.3 w/v % CaCl₂ in DI water) for 5 min. The cell-laden hydrogels were transferred to wells of well-plate (Corning) in the relevant culture media containing 1 w/v % TG. After curing in an incubator for 12 hours, the media were replaced with fresh culture media and changed every 2-3 days. Additionally, using the same protocol, hMSC-laden red-fluorescent BC-DN hydrogels were prepared to visualize the cellular behavior.

### Cell Distribution and Leaching Evaluations

To identify cell distribution and cell leaching, green cell-tracker-labeled NIH/3T3 were encapsulated in red-fluorescent hydrogels. Cell-tracker in DMSO was diluted with DPBS (0.4 μg mL⁻¹). The NIH/3T3 cells pellet was resuspended, incubated in cell-tracker solution at 37° C. for 20 min, and centrifuged/washed with DPBS three times before encapsulation. Both cell-laden DN and BC-DN hydrogels (6.2×10⁶ cells mL⁻¹) were prepared using cell-tracker-labeled cells to evaluate the cell microphase.

Besides, a cell-laden porous DN hydrogel was prepared by selectively leaching out the biopolymer-poor phase. In brief, emulsion bioink (5.6×10⁶ cells mL⁻¹) containing 1 w/v % TG was cast in PDMS mold and gelled at 37° C. for 30 min. The soft films were transferred into wells with DMEM to leach out the Alg-Gel and PVA in biopolymer-poor phase in 30 min. After replacing the medium with DMEM supplemented with 1 w/v % TG, the samples were incubated overnight at 37° C. Later, the samples were soaked in 0.3 w/v % CaCl₂ before switching to a fresh medium. The cell distribution in hydrogels was captured by a (confocal) fluorescence microscope, and cell density was quantified with ImageJ.

### Cellular Behavior Characterizations

### Cell Viability:

Viability was measured by the Live/Dead viability/cytotoxicity kit according to the manufacturer's instructions. Briefly, cell-laden hydrogels collected on days 1, 3, and 7 were washed with DPBS twice, followed by the addition of calcein-AM (2 mM) and ethidium homodimer-1 (4 mM) in DPBS. After incubating at 37° C. for 20 min, the samples were washed twice with DPBS and observed under a fluorescence microscope. Percentages of viable cells were analyzed based on triplicate (n=3) results using ImageJ.

### Cellular Metabolic Activities:

Metabolic behavior was evaluated by cell proliferation assay according to the manufacturer's instructions. Briefly, the cubic samples were incubated with the MTS assay (1:5 dilution in a relevant cell culture medium) for 4 hours in the dark. Subsequently, the absorbance at 490 nm was recorded using a microplate reader (Molecular Devices) (n=2-3). Normalized results based on day 1 were reported.

### F-Actin Staining

Samples collected at different culture periods were fixed with 10% formalin for 15 min and permeabilized with 0.3 v/v % triton X-100 for 20 min at room temperature, followed by staining with Alexa Fluor™ 488-phalloidin (1:500 dilution in DPBS) overnight at 4° C. The nuclei were counterstained with the DAPI (1:1,000 dilution in DPBS) for 15 min at room temperature. The samples were washed with DPBS twice before each step and further washed in DPBS overnight at 4° C. Fluorescence microscope images were taken using a confocal fluorescence microscope.

### Mechanical Durability:

The influence of cellular activities on hydrogel mechanical properties was assessed by compression test of hMSC-laden BC-DN hydrogels. Both original and mechanically boosted (treated with 3 w/v % CaCl₂ for 5 min) cube samples collected on days 1, 7, and 14 were compressed at a rate of 50% min⁻¹. At least two specimens (n=2-3) were tested for each sample.

### Cartilage Biofabrication and Characterizations

### Biofabrication

We fabricated chondrogenic hMSC-laden hydrogels as engineered neo-cartilage. Briefly, hMSCs (passage 5, 6.0×106 cells mL⁻¹) encapsulated in BC-DN hydrogels were cultured in the MSC growth medium for 7 days and then switched to chondrogenic DMEM, supplemented with 10 v/v % FBS, 1 v/v % Anti-Anti, 40 μg mL⁻¹ of L-proline, 10 μg mL⁻¹ of ITS-supplement, 50 μg mL⁻¹ of L-ascorbic acid, 110 μg mL⁻¹ of sodium pyruvate, 100 nmol L-1 of dexamethasone and 10 ng mL⁻¹ of TGF-β3. All mediums were changed every 3 days. The samples collected on days 7, 14, and 21 after differentiation were fixed with 10% formalin for further study.

Cellular assays: F-actin staining was performed using the aforementioned protocol. For co-staining of collagen X, the samples after F-actin staining were washed with 1 v/v % BSA and treated with blocking buffer (5 v/v % BSA and 0.2 v/v % triton X-100 in DPBS) for 2 hours at room temperature, stained with primary antibody of anti-collagen X antibody (1:200 dilution in blocking buffer) overnight at 4° C., and incubated with secondary antibody (Alexa Fluor™ 594-goat anti-mouse IgG, 1:500 dilution in blocking buffer) at 37° C. for 2 hours with DPBS washing twice after each step. Individual immunostaining of aggrecan was performed using a primary antibody of anti-aggrecan antibody (1:200 dilution in blocking buffer), a secondary antibody (Alexa Fluor™ 488-goat anti-rabbit IgG, 1:500 dilution in blocking buffer), and counterstained with DAPI (1:1,000 dilution in DPBS). Confocal microscope images were taken after further washing overnight at 4° C. For histological staining, the samples were sliced (5-μm sections) in paraffin blocks. The deparaffinized slices were stained with Alcian blue for glycosaminoglycan, and Masson's trichrome for collagen deposition.

Quantitative Reverse Transcription-Polymerase Chain Reaction (qRT-PCR) Experiments and Data-Processing

The gene expressions of chondrogenesis-specific genes, including SOX9, AGCAN, COL1A1, COL10A1, COMP, and ELASTIN, were performed on a qRT-PCR. The chondrogenic samples for both hMSC-laden DN and BC-DN hydrogels were collected for gene expression evaluations on days 7, 14, and 21 of differentiation. The samples were digested in collagenase type IV (1 mg mL⁻¹ in DPBS) at 37° C. for 40 min, trypsinized in trypsin-EDTA, and centrifuged to release the cells. Then, total RNAs were isolated using RNA easy mini kit (QIAGEN), and the first-strand cDNA was synthesized using the QuantiTect Reverse Transcription Kit (QIAGEN) according to the manufacturer's instructions. PCR analysis was carried out with a QuantStudio 5 Real-Time PCR instrument (Thermo Fisher) using the standard thermal cycling conditions. The cycle threshold (Ct) values were recorded for each gene (n=2-3). To analyze the data, each targeted gene was normalized by the housekeeping gene (GAPDH) to obtain the difference in Cₜ (ΔCₜ, =Ct, _{gene}−C_{t, GAPDH}). The relative gene expressions for different differentiation periods were further normalized against weak-1 of differentiation using the 2^{-ΔΔCt} algorithm (ΔΔCt=ΔCt, sample−ΔC_{t, week1}). The relative expression for the specific genes versus different times can be plotted to study the time-dependent differentiation progress. In addition, the relative gene expressions in hMSC-laden DN and BC-DN hydrogels were compared to identify the role of cellular activities on cellular functions.

### Mechanical Measurement

Compression test was conducted on engineered neo-cartilage at various differentiation periods with a mechanical testing machine (100-N load cell, Instron 3342) using a compressive rate of 50% min⁻¹. Two specimens (n=2) were tested for each sample. Using the aforementioned protocol, the crashed samples were instantly fixed with 10% formalin for F-actin staining.

### Engineered Vessel Biofabrication and Characterizations

### Biofabrication

HUVSMC-laden AG/PVA (6/4) emulsion bioink (1.0-1.5×10⁷ cells mL⁻¹) was used for coaxial bioprinting of cellular tubes following a similar protocol for acellular printing. The support bath consisting of 0.01 w/v % CaCl₂) in SMC medium was used to collect the cell-tubes. Later, the cell-tubes were transferred to SMC medium supplemented with 1 w/v % TG for incubation overnight at 37° C. The medium was replaced with the fresh medium and further changed every 3 days.

### Cellular Assays

Live/dead assays were conducted for cell tubes on days 1, 3, and 7 of culture. Cell tubes collected on days 7, 14, and 21 were fixed with 10% formalin for F-actin staining. On day 21 of bioprinting, HUVECs were post-seeded in the lumen of hollow tubes by perfusing 150 μL of HUAEC suspension (2-3×10⁷ cells mL⁻¹) followed by co-culture in a common medium (SMC medium/EGM-2 medium=4/6, v/v) for an additional week. The resulting engineered vessels were fixed with 10% formalin for immunostaining. The samples were stained with primary antibodies of anti-VE-cadherin and anti-α-SMA (1:200 dilution in blocking buffer) overnight at 4° C., incubated with secondary antibodies (Alexa Fluor™ 488-goat anti-rabbit IgG and Alexa Fluor™ 594-goat anti-mouse IgG, 1:500 dilution in blocking buffer) at room temperature for 2 hours, followed by counterstaining with DAPI (1:1,000 dilution in DPBS) for 15 min at room temperature. The samples were washed twice with DPBS after each step and further washed overnight at 4° C. before examining under the confocal microscope.

### Mechanical Measurement

Tensile tests were conducted on bioprinted cell-tubes (approximately 1,500-μm outer diameter, 400-μm wall thickness) using a mechanical testing machine (100-N load cell, Instron 3342). A gauge length of 2-4 mm was adopted. Pristine and boosted (3 w/v % CaCl₂)) cell-tubes wrapped with paper tape on the two ends were pulled to break (n=2-3). Using the aforementioned protocol, the fractured samples were instantly fixed with 10% formalin for F-actin staining.

### Ex Vivo Suture Tests

Discarded human vascular tissues were harvested under the protocol by the institutional review board at the Brigham and Women's Hospital, for which informed consent did not apply. (Bio)printed acellular BC-DN hydrogel tubes (2-3 cm in length, 4-5 mm in out-diameter, 1.3-mm wall thickness) were anastomosed with human veins (4-5 mm inner-diameter) using a running 6-0 monofilament non-absorbable polypropylene suture (PROLENE™, Ethicon) or 4-0 monofilament absorbable poliglecaprone 25 suture (MONOCRYL™, Ethicon). The anastomosed conduits with four stitches were perfused with fluorescent microbeads (Createx Colors) suspended in DPBS. The anastomosed conduits connected by two stitches were pulled apart at a rate of 100% min⁻¹, and the peak force was recorded as the suture-retention strength (n=3-5). A cyclic test was performed on the sutured conduits by loading-unloading with a maximum strain of 20% at a rate of 1,000% min⁻¹.

### Example 2: Microfluidic Bioprinting of Tough Hydrogel-Based Vascular Conduits for Functional Blood Vessels

Herein, we present a stretchable DN hydrogel (bio)ink system for microfluidic bioprinting of small-diameter vascular conduits that favorably recapitulated structural and biological functions of their native counterparts. The (bio)ink prepolymer, composed of sodium alginate and gelatin (or gelatin methacryloyl (GelMA)), was crosslinked by calcium chloride (CaCl₂) and microbial transglutaminase (mTG), respectively, generating a DN hydrogel with excellent mechanical properties and good biocompatibility. Using microfluidic bioprinting, two distinct approaches were adopted to fabricate venous and arterial engineered vessels closely resembling their native counterparts. Specifically, venous conduits were generated by (bio)printing mono-layered hydrogel tubes, followed by seeding human umbilical vein ECs (HUVECs) in the lumens and human umbilical vein smooth muscle cells (HUVSMCs) on the outer surfaces. In contrast, arterial conduits were generated by direct bioprinting of the outer human umbilical artery smooth muscle cell (HUASMC)-encapsulated layer and inner hydrogel layer followed by seeding human umbilical artery ECs (HUAECs) in the lumens. Both venous and arterial conduits exhibited key properties of blood vessels, including high stretchability, apparent perfusability, and barrier performance. More importantly, the arterial conduits displayed constriction and dilation responses to vasoconstrictor and vasodilator, respectively. Furthermore, we demonstrated the applicability of these vascular conduits for studying diseases and drug testing in vitro, by infecting them with pseudotyped severe acute respiratory syndrome coronavirus-2 (SARS-CoV-2) viral particles (pCoV-VPs) expressing spike proteins and subjecting them to treatment with antiviral drugs. Similarly, we conducted ex vivo vascular anastomoses by connecting the vascular conduits with a human popliteal vein or a mouse aorta, as well as preliminary in vivo vascular anastomosis with a mouse vena cava, illustrating the potential of the 3D-(bio)printed vascular conduits as vascular grafts in the future.

### Results and Discussion

### Design and Mechanics of Tough DN Hydrogels

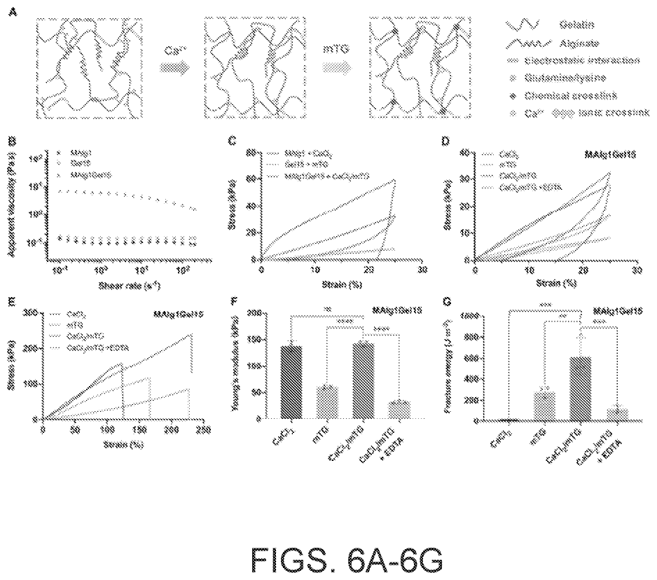

Central to the (bio)printing of biomimetic vessels is to develop a cytocompatible (bio)ink with suitable rheological properties, cell-benign crosslinking, and vessel-relevant mechanical performances. Tough hydrogels that possess excellent mechanical properties are highly desired for such a purpose. Recent advances in DN hydrogels provide a general method to fabricate hydrogel constructs with unprecedented mechanical properties and good biocompatibility (X. Zhao, et al., Chem Rev 121, 4309-4372 (2021)). With these in mind, we engineered a set of natural polymer-based DN hydrogels consisting of alginate physically crosslinked by calcium and gelatin covalently crosslinked by mTG (FIG. 6A). Gelatin can form covalent crosslinks between glutamine and lysine groups upon treatment with mTG and intrinsically possesses arginylglycylaspartic acid (ArgGlyAsp; RGD) peptide sequences that promote cell adhesion and proliferation (N. Contessi Negrini, et al., ACS Biomaterials Science & Engineering 7, 4330-4346 (2021)). It should be noted that mTG is a United States Food and Drug Administration (FDA)-approved enzyme to achieve cell-benign crosslinking (C. W. Yung, et al., Biomedical Materials Research Part A 83, 1039-1046 (2007)). The ionically crosslinked alginate, also biocompatible, enables effective energy-dissipation for enhancement of mechanical properties (J. P. Gong, et al., Soft Matter 6, 2583-2590 (2010)). Besides the excellent biocompatibility, another prominent advantage of using these natural polymers is the existence of electrostatic interactions within the selected components, enabling tunable rheological properties of the bioink. For example, due to the strong electrostatic interactions between alginate and gelatin, the hybrid (bio)ink containing 1% medium-viscosity alginate (MAlg) and 15% gelatin exhibited an apparent viscosity (7 Pa·s), over 40 times higher than those of the individual components (approximately 0.14-0.17 Pa·s) at the shear rate of 0.1 s⁻¹ (FIG. 6B).

The mechanics of the DN hydrogels were studied by tensile tests. We first investigated the mechanical properties of the individual components. FIG. 6C shows the loading-unloading stress-strain curves of the 1% MAlg (MAlg1) hydrogel and the 15% gelatin (Gel15) hydrogel separately, as well as the hybrid (bio)ink containing these two components together (MAlg1Gel15), to a maximum strain of 25%. The MAlg hydrogel physically crosslinked by 2% CaCl₂ and the gelatin hydrogel covalently crosslinked by 2% mTG showed moduli of 242.1 kPa and 34.6 kPa, respectively. The physically crosslinked alginate exhibited a large hysteresis ratio (78%) and a large irreversible strain (22%). Although the gelatin hydrogel showed a low hysteresis ratio of 9% and a low strain set (1%), its mechanical property was weak due to the lack of energy-dissipation. Interestingly, the MAlg1Gel15 hydrogel, crosslinked with 2% CaCl₂ and 2% mTG (CaCl₂/mTG), exhibited a lower Young's modulus (142.8 kPa) than that of the pure MAlg1 hydrogel, likely attributed to the suppression of alginate crosslinking by the electrostatic interactions between alginate and gelatin chains. The MAlg1Gel15 DN hydrogel possessed higher strength and stretchability than those of single-component hydrogels due to the two intertwined networks. The physically crosslinked alginate network tremendously contributed to energy-dissipation in the DN hydrogel, as indicated by the hysteresis ratio of 49%. The DN hydrogel also showed a low irreversible strain or strain set (5%) during loading-unloading cycles using a maximum strain of 25%, due to the covalently crosslinked gelatin network. These results confirmed that the alginate-gelatin hydrogel is a DN hydrogel with enhanced mechanical performance.

We further analyzed the role of physical and chemical crosslinking in the mechanical properties of DN hydrogels. The DN hydrogels with physical, chemical, and dual crosslinking were fabricated. To decouple the contributions of physical and chemical crosslinking, a chelating agent, ethylenediaminetetraacetic acid (EDTA), was used to selectively cleave the physical crosslinking in the DN hydrogels. As shown in FIG. 6D, the dual-crosslinked DN hydrogel manifested lower energy-dissipation (smaller hysteresis loop) and higher strain-recovery than those of hydrogels possessing only physical crosslinking. The hydrogel with only chemical crosslinking by mTG also exhibited an obvious hysteresis loop (hysteresis ratio of 20%), due to energy-dissipation by electrostatic interactions between alginate and gelatin. The dual-crosslinked DN hydrogels possessed the highest tensile strength (197.7 kPa) and tensile strain (207.3%) compared with the single-network hybrid hydrogels (FIG. 6E). Compared with conventional DN hydrogels with independent crosslinking of each component, the ionic crosslinking and enzyme crosslinking in our design could influence each other in the resulting DN hydrogel, as indicated by the similar modulus of the DN hydrogel to that of the hydrogel with only ionic crosslinking (FIG. 6F). This result was also supported by the lower Young's modulus of the EDTA-treated hydrogels than that of hydrogels only crosslinked by mTG, because the fast and dense ionic crosslinking of alginate could likely decrease the enzyme crosslinking degree of gelatin due to high network-hindrance (O. Chaudhuri, et al., Nat. Mater. 15, 326-334 (2016)).

Owning to effective energy-dissipation and enhanced stretchability, the dual crosslinked DN hydrogel offered a high toughness of 616.3 J·m², exceeding that of the physically crosslinked single-network hydrogel (14.9 J·m⁻²) and the chemically crosslinked single-network hydrogel (279.5 J·m⁻²) (FIG. 6G). After removal of the physical network of the DN hydrogel by EDTA, the fracture energy reduced to merely 121.5 J·m⁻², highlighting the crucial role of the alginate physical network in energy-dissipation in the tough DN hydrogels. Although the alginate network can be damaged during deformation, the stretchy gelatin network would maintain the material integrity.

### Microfluidic Coaxial Extrusion (Bio)Printing and Mechanical Properties of (Bio)Printed Vascular Conduits

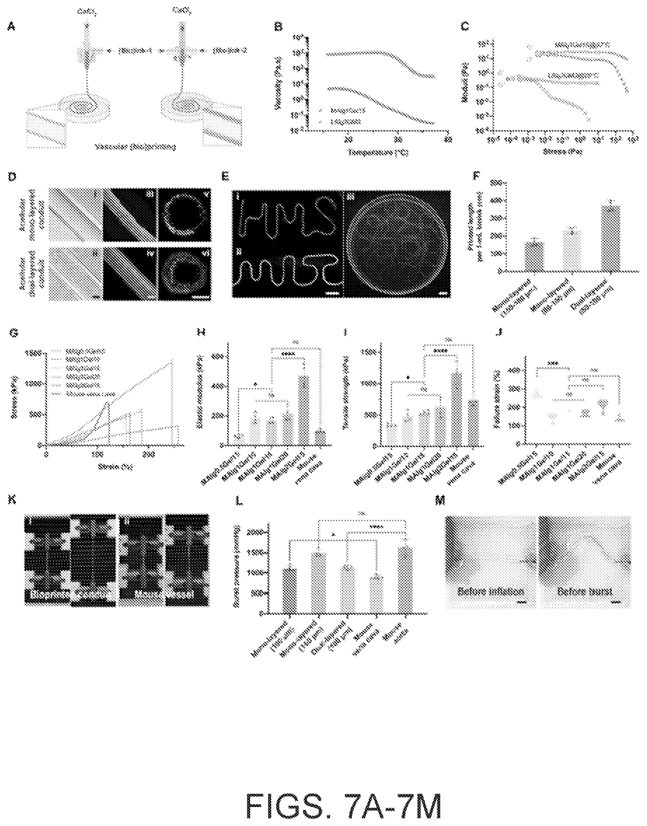

After understanding the mechanical properties, the tough DN hydrogel (bio)inks were used for the high-throughput fabrication of vascular conduits by coaxial extrusion (bio)printing. Two types of multichannel coaxial extrusion systems featuring two or three channels were utilized to produce mono-layered or dual-layered vascular conduits, respectively. CaCl₂ solution flowing in the core layer acted as the physical crosslinker for alginate in the hybrid (bio)ink (FIG. 7A). The (bio)printed conduits were subsequently treated with the CaCl₂/mTG solution for post-printing curing. (Bio)inks with different formulations were exploited to (bio)print tubes with widely tunable mechanical properties. Before setting the (bio)printing parameters, the rheological properties of two typical (bio)inks were measured. In FIG. 7B, the complex viscosity of the (bio)ink MAlg1Gel15 decreased two orders of magnitude with a transition temperature at approximately 31° C. Above the transition temperature, the (bio)ink transformed from solid to liquid, as indicated by the plateau loss modulus exceeding the storage modulus. At a lower temperature (such as 30° C.), the viscosity and shear stress of the MAlg1Gel15 (bio)ink became very high and thus not suitable for microfluidic extrusion (bio)printing. Meanwhile, another DN hydrogel (bio)ink containing 2% low-viscosity alginate (LAlg) and 3% GelMA (LAlg2GM3), which was found to be suitable for cell encapsulation, was also measured. LAlg2GM3 possessed a much higher viscosity than individual components, again due to the electrostatic interactions between the components. However, its viscosity was very low at both room temperature and 37° C. due to the relatively low polymer concentration and weak electrostatic interactions between alginate and GelMA. The rheological properties of various (bio)inks could be fine-tuned to facilitate coaxial extrusion (bio)printing by adjusting the temperature. For example, the MAlg1Gel15 (bio)ink with a transition temperature of 31° C. could be heated to 37° C., yielding a low viscosity fluid (FIG. 7C). In comparison, the LAlg2GM3 (bio)ink needed to be cooled down to increase the viscosity and impart shear-thinning for (bio)printing at room temperature. Both of these (bio)inks showed low yield stresses (crossover of G′ and G″) at the (bio)printing temperature range, enabling (bio)ink-extrusion at low shear stresses (FIG. 7C).

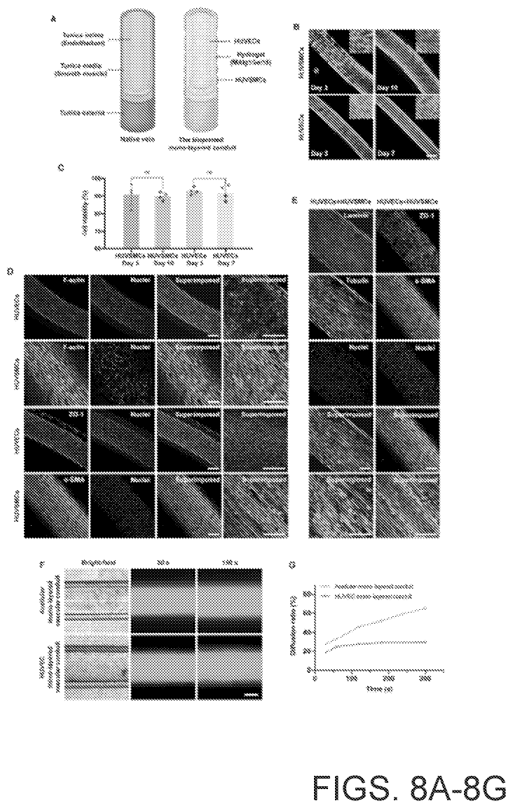

During the coaxial extrusion process, the flow of CaCl₂ in the core allowed for in situ physical crosslinking of the alginate component of the (bio)ink, serving to maintain the tubular shape of the conduits (J. Li, et al., Journal of Materials Science 51, 5791-5801 (2016)). In addition, a bath containing CaCl₂/mTG solution was used to further crosslink the (bio)printed tubes. Together with properly set (bio)printing parameters, high-quality dual-crosslinked hollow tubes could be obtained. As shown in FIG. 7D, the (bio)printed mono- and dual-layered conduits displayed uniform sizes and smooth surfaces. Coaxial extrusion (bio)printing has several prominent advantages in fabricating vascular conduits. First, the method would produce structurally relevant tubes with tunable diameters and wall thicknesses for both mono- and dual-layered conduits (FIG. 8D). In addition, this approach enabled the high-throughput fabrication of long, continuous tubes (FIG. 7E). For example, up to 19 m of acellular conduits could be continuously extruded without clogging the nozzle in a single (bio)printing session. Moreover, coaxial (bio)printing enabled efficient conduit-production with minimized (bio)ink waste, ideal for cost-effective and large-scale fabrication of conduits. As a demonstration, 1 mL of the MAlg1Gel15 (bio)ink could be used to generate as long as 165 cm of a mono-layered conduit with 150-180 μm of wall thickness, and a 232.9-cm dual-layered conduit with the inner wall thickness of 80-100 μm (FIG. 7F).

(Bio)inks with different formulations were adopted to produce small-sized vascular conduits of widely tunable mechanical properties. The mechanical properties of the (bio)printed mono- and dual-layered conduits were studied and compared with mouse vena cava and aorta of similar sizes. FIG. 7G shows the stress-strain curves of the mono-layered conduits (bio)printed using different (bio)inks containing various MAlg contents (0.5-2%) and gelatin contents (10-20%). Young's modulus and tensile strength of the (bio)printed tubes are very sensitive to content of MAlg instead of gelatin (FIGS. 7H-J). For example, for printed DN hydrogels consisting of 15% Gelatin, Young's modulus can increase from 65.3 kPa to 472.0 kPa with increasing MAlg content from 0.5 to 2%. The (bio)printed DN hydrogel tubes containing an alginate content of 0.5% were mechanically weak, while the stiffness and strength of the (bio)printed tubes with 2% MAlg were much higher than those of the mouse vein. In contrast, the DN hydrogel tubes consisting of 1% MAlg possessed mechanical properties matching the native mouse vena cava's tensile modulus, failure strain, and ultimate tensile strength. However, no significant difference in mechanical strength was observed among the 10-20% gelatin tubes with fixed alginate. In terms of mechanical properties, the MAlg1Gel15 hydrogel-based tubes had the strength of 538.0 and failure strain of 183.9%, similar to those of mouse vena cava of 738.1 kPa and 134.7%, respectively. FIG. 7K shows that the MAlg1Gel15 tubes and mouse vena cava could be stretched to similar lengths. Therefore, considering both mechanical properties and printability, the MAlg1Gel15 (bio)ink was selected as the optimal (bio)ink for the (bio)printing of the conduits.

Notably, the DN hydrogel-based tubes also exhibited good physiologically mechanical stability. The MAlg1Gel15 tubes maintained a stable stiffness in cell culture medium (e.g., smooth muscle cell medium) for up to 2 weeks evaluated. The satisfactory physiological stability of the current DN hydrogels could be attributed to the dense ionic crosslinking by the higher guluronate/mannuronate blocks ratio of the used alginate (J. L. Drury, et al., Biomaterials 25, 3187-3199 (2004)).

We also (bio)printed dual-layered vascular conduits to mimic the artery with two different layers. The mechanically robust MAlg1Gel15 hydrogel was used as the inner layer to provide mechanical support, while the weaker hydrogel (bio)inks containing 3% GelMA and different types and concentrations of alginates were used for bioprinting the outer layer to support cell growth. When using MAlg from 1% to 0.5%, Young's modulus of the dual-layered tubes decreased from 208.5 kPa to 110.2 kPa. To further reduce the tube's stiffness, 2% LAlg and 3% GelMA were formulated as the (bio)ink (LAlg2GM3) to (bio)print the outer layer of the dual-layered conduits. The resultant tubes possessed a low Young's modulus of 63.4 kPa while retaining a high stretchability (240.4%), falling in the similar range of the mouse aorta (58.9 kPa and 158.1%, respectively).

Besides, we measured the burst pressures of the (bio)printed mono- and dual-layered conduits to illustrate their similarities to native vessels. According to the Laplace's law, burst pressure is positively correlated with the wall thickness (S. K. Burke, et al., J Cardiovasc Pharmacol 67, 305-311 (2016)). The mouse vena cava and aorta with similar dimensions to the (bio)printed tubes were used as benchmarks. These vascular conduits were highly inflatable and could be tied by 5-0 sutures to the metal connectors. The mono-layered conduits (100 μm in wall thickness) exhibited a slightly higher burst pressure (1,113.1 mmHg) than that of the mouse vena cava (897.1 mmHg) with a similar wall thickness (FIG. 7L). The burst pressure increased to 1,497.6 mmHg at a wall thickness of 160 μm. The 100-μm wall thickness of the dual-layered conduits possessed a burst pressure of 1,137.8 mmHg, which was slightly lower than that of the mouse aorta (1,630.9 mmHg). The above results suggested the (bio)printed DN hydrogel conduits using our optimized (bio)inks were structurally and mechanically relevant to the native vessels to a reasonable extent.

### Perfusability and Permeability of (Bio)Printed Conduits

Perfusability and selective permeability, the basic functions of blood vessels, allowing for the flow and diffusion of small molecules through the wall while retarding or blocking the transport of macromolecules (J. Gavard, et al., Cell Adh Migr 7, 455-461 (2013)). In the majority of the previously reported studies, including those reported by us (Q. Pi, et al., Advanced materials (Deerfield Beach, Fla.) 30, e1706913 (2018)), (bio)printed hydrogel-based vascular conduits are vulnerable and mechanically weak. These vascular conduits would oftentimes fail to withstand physiological pressure exerted by the flow, limiting their in vitro and in vivo applications. This fact has been deemed as a common drawback for many of these coaxially extruded hollow conduits (W. Liu, et al., Biofabrication 10, 024102 (2018)).

Nevertheless, the (bio)printed tubes developed in this study were tough enough to withstand surgical knots and amenable to insertion and fixation of blunt metal needles, which are important for post-bioprinting studies and applications. To assess perfusability and permeability, these (bio)printed tubes were connected to metallic connectors in a bioreactor and maintained a closed circulation system using a peristaltic pump. The fluorescein isothiocyanate (FITC)-conjugated dextran (FITC-Dex) of two different molecular sizes, 3-5-kDa and 150-kDa, representative of small molecules and macromolecules, respectively, were then perfused through the (bio)printed tubes. FITC-Dex (150-kDa) could barely diffuse through the wall of the mono-layered conduit during the 24-h perfusion period, whereas the 3-5-kDa FITC-Dex rapidly penetrated in a short time and was further distributed throughout the bioreactor reservoir in the following hours as indicated by the remarkable differences in fluorescence intensities in the reservoir. Similar diffusion profiles were observed in the dual-layered conduits for both 3-5-kDa FITC-Dex and 150-kDa FITC-Dex. A 16-fold reduction was measured in the permeability of 150-kDa FITC-Dex as compared to that of 3-5-kDa FITC-Dex in mono-layered conduits, whereas the value for the dual-layered conduits dramatically decreased from 7.2×10⁻³ to 0.6×10⁻³ cm·s⁻¹. These results suggested the selective permeability of molecules across the walls of the (bio)printed conduits. We were able to maintain the perfusion of FITC-Dex (150-kDa) or medium through the vascular conduits for up to 3 days tested without any leakage, demonstrating their potential for long-term applications under physiological circulation conditions. An ideal vascular conduit should support the flow of blood and withstand the pressures exerted by the blood flow immediately upon implantation in vivo (S. Pashneh-Tala, et al., Tissue engineering. Part B, Reviews 22, 68-100 (2016)). Therefore, we further assessed the perfusion of red blood cells (RBCs) through a long mono-layered conduit without noticeable rupture or leakage, illustrating that the (bio)printed conduits may possess the potential to receive and withstand the pressures exerted by the blood flow while maintaining structural integrity.

### Generation of Venous Conduits

The venous blood vessel wall consists of the inner endothelium composed of ECs, the middle muscular layer composed of smooth muscle cells (SMCs) and elastic tissue, and an outer fibrous connective tissue layer. Veins and venules have thinner muscular walls than arteries and arterioles, largely because the pressures and rates of blood flow in veins and venules are much lower (FIG. 8A). To recreate the native vein-like venous conduit with functional endothelial and muscular layers, HUVSMCs were first seeded on the outer surface of the (bio)printed mono-layered conduit. After 3 days of culture under static conditions at 37° C., a compact layer of HUVSMCs was formed across the entire outer surface of the conduit. HUVECs were then perfused and allowed to attach in the inner lumen of the conduit already having the outer smooth muscle layer. After incubation of the conduit under similar culture conditions for 7 additional days (thus a total of 10-day culture), a hollow venous conduit with a layer of confluent endothelium in the lumen and a thin, smooth muscle sheath on the outer surface was eventually constructed for further characterizations. The viabilities of HUVSMCs and HUVECs were assessed separately at selected time points. As shown in FIG. 8B-C, the viability values of HUVSMCs were 90.9% at day 3 and 89.9% at day 10, while those of HUVECs were 92.9% at day 3 and 92.0% at day 7, respectively.

The proliferation and morphologies of both HUVECs and HUVSMCs in the (bio)printed and cellularized venous conduits were assessed by F-actin staining. The expression of tight junction proteins, zonula occludens-1 (ZO-1), by HUVECs indicated the formation of intercellular junctions between the cells necessary for the proper functioning of the endothelial layer. Similarly, expression of α-smooth muscle actin (α-SMA), an actin isoform that plays a role in contractility of vascular SMCs, by HUVSMCs revealed the successful formation of a muscular layer on the outer surface of the conduit (FIG. 8D). In addition, the expression of laminin by HUVECS indicated that the HUVECs were actively synthesizing laminin, which represents one of the major structural components of the basement membrane and is essential for adhesion of ECs to the basement membrane and shear stress-response of blood vessels. Besides α-SMA, HUVSMCs were also found to express tubulin, another marker protein of the contractile phenotype of vascular SMCs (FIG. 8E). Expression of these markers by HUVECs and HUVSMCs confirmed the successful formation of venous conduits with functional endothelium covering the inner wall of the lumen and a muscular layer across the outer surface.

Importantly, endothelium plays a pivotal role as a vascular barrier in controlling the extravasation of biomolecules, nutrients, and cells. This barrier function is proven to be regulated by ECs lining the luminal surfaces of the vessels but not disturbed by SMCs in homeostasis (Andrique, et al., Science advances 5, eaau6562 (2019)). To further evaluate the barrier function of the venous conduits, we assessed the permeability of FITC-Dex (3-5 kDa) using the endothelialized venous conduits. The acellular conduits of the same size were used as the control. FITC-Dex was perfused through acellular conduits and endothelialized conduits, separately. Diffusion ratio, which was measured as the ratio of the grayscale area of diffused FITC-Dex in a fixed microscope field at a selected time point, reflected the permeating speed through the conduit. As shown in FIG. 8G-H, FITC-Dex rapidly permeated into the walls of the acellular conduits and then penetrated out from the lumens. In contrast, the presence of the HUVEC layer effectively delayed the diffusion speed of the molecule. Accordingly, these results confirmed the barrier function of the endothelialized 3D-(bio)printed venous conduits.

### Generation of Arterial Conduits

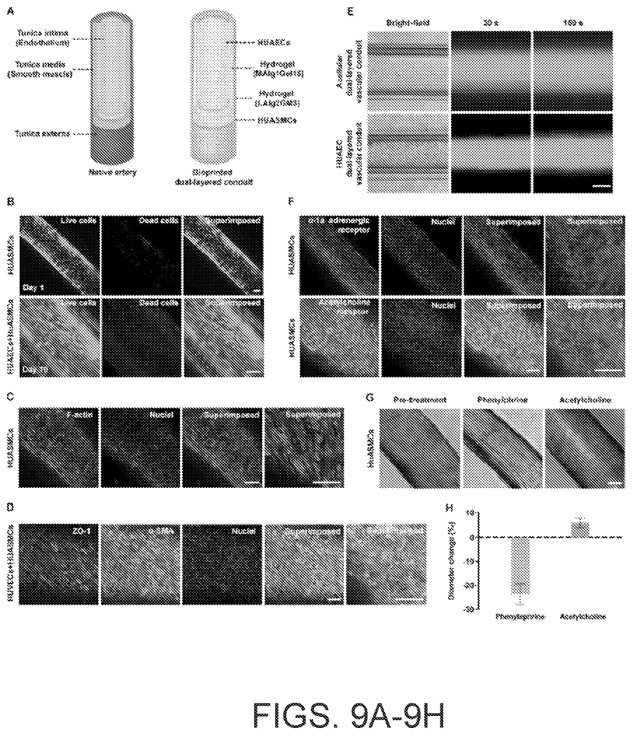

Arteries and arterioles have relatively thick muscular walls (FIG. 9A) because they have to withstand higher blood pressures. Therefore, the arterial conduits with thicker muscular walls were generated by bioprinting of dual-layered vascular conduits comprised of the outer HUASMC-encapsulated layer and inner hydrogel layer. HUAECs were subsequently seeded in the lumen of the inner wall after the maturation of the HUASMCs. While the same ink formulation as used for (bio)printing the venous conduits (MAlg1Gel15) was used for generating the inner robust hydrogel layer, the cell-friendly outer-layer bioink was optimized to enable desirable cellular behaviors. The initial elastic modulus of a hydrogel has a significant impact on the morphology and proliferation of cells embedded in it. Generally, materials with lower stiffness values and larger mesh sizes are beneficial for cell spreading and proliferation (R. Goldshmid, et al., Biomaterials Science & Engineering 3, 3433-3446 (2017)). Accordingly, the mechanical properties of the (bio)printed mono-layered hollow tubes using various (bio)ink formulations consisting of different combinations of alginate and gelatin or GelMA were first tested. For a fixed GelMA content of 3% (GM3), the initial stiffness of the tubes was influenced by both alginate content and type. The initial Young's moduli of the samples containing 2% LAlg were below 100 kPa. In comparison, the MAlg1GM3 tubes produced with MAlg possessed a much higher Young's modulus (265.3 kPa).

We further assessed the changes in their physiological stability in SMC culture medium. The modulus of the LAlg2Gel3 tubes dramatically dropped from 62.0 kPa to 29.6 kPa after 4 h of incubation in the SMC medium. Because the physical network LAlg can be rapidly degraded via ionic-exchange by the medium. The stable plateau modulus for extended treatment time was attributed to the chemically crosslinked network of gelatin. After medium-treatment, the mechanical property of LAlg2GM3 tubes was not shown because the material became too soft to perform tests. Nevertheless, the above results highlighted the obvious mechanical property-changes of LAlg-based hydrogels when subjected to incubation in a buffer solution or medium. In contrast, the inner-layer material of MAlg1Gel15 as mechanical support of the tubes showed good physiological stability, suggesting the type of alginate plays a key role in determining the stability of the resulting hydrogels.

The cellular behaviors in the above bioinks were further assessed. Bioinks composed of 3% GelMA and LAlg at up to 2% enabled spreading of HUASMCs, as indicated by F-actin staining results. By contrast, the bioinks containing MAlg inhibited the growth of HUASMCs even at alginate content as low as 0.5%. It was also noted that the bioinks containing LAlg at less than 2% exhibited poor printability. Accordingly, LAlg2GM3 was selected as the optimal bioink with good cellular behaviors and printability for bioprinting the outer layers of the conduits with HUASMCs encapsulated. The bioprinted HUASMC-laden conduits were incubated at 37° C. for at least 14 days until a tight volume of HUASMCs was formed within the outer layers. FIG. 9B represents the viability of the bioprinted HUASMCs at day 3 and day 10 of bioprinting. F-actin staining of the bioprinted HUASMCs demonstrated the evenly spreading HUASMCs in the arterial conduits (FIG. 9C).

After approximately 14 days, HUAECs were further seeded in the lumen by perfusing the cell suspension, following the similar procedure as used for seeding the venous conduits. After additional 7 days of culture, hollow arterial conduits were obtained with a layer of endothelium layer in the lumen and a relatively thick smooth muscle sheath on the outer surface. The average thickness of the HUASMC layers in the arterial conduits was found to be 55 μm, which was noticeably thicker than that in the venous conduits (~20 μm). The expressions of ZO-1 by HUAECs and α-SMA by HUASMCs revealed the formation of endothelial layer in the lumen wall and compact smooth muscle layer on the outer surface of the conduit (FIG. 9D). Similarly, the barrier function of arterial conduits was also confirmed by perfusing FITC-Dex (3-5-kDa). The permeability of FITC-Dex through the endothelialized arterial conduits suggested that the presence of the compact HUAEC layer obviously decelerated the transport rate of these fluorescence molecules when compared to the control without the endothelium (FIG. 9E).

Blood vessels, particularly the arteries and arterioles, constantly receive a variety of vasoconstrictor- and vasodilator-stimuli wherein SMCs in the muscular layer respond to these stimuli causing constriction or dilation to regulate the vascular tone and hence, blood flow and blood pressure (Davis, Michael, et al., Physiological Reviews 79, 387-387 (1999)). Vasoconstriction is narrowing of lumen as a result of contraction of the SMC-layer, whereas vasodilation is widening of lumen resulting from relaxation of SMCs (M. A. Hill, et al., Trends Pharmacol Sci 30, 363-374 (2009)). To evaluate the contractility and thus phenotype of HUASMCs in the muscle layer of our bioprinted arterial conduits, phenylephrine, one of the potent vasoconstrictors (K. F. Franzen, et al., Cells 10, (2021)), was applied to the arterial conduits. Since phenylephrine induces vasoconstriction through α-adrenergic receptor, prior to contractility evaluations, we assessed the expressions of α-1a adrenergic receptor by arterial SMCs in arterial conduits (FIG. 9F). al-adrenoceptor stimulation with phenylephrine (10 μM) elicited a notable reduction of the diameter of the arterial conduits, reflecting the constriction of arterial SMCs within the muscle layer of arterial conduits (FIGS. 9G-H). Similarly, studies have shown that acetylcholine, a vasodilator, would relax contractions of vascular smooth muscle induced by phenylephrine, mediated by the M3 muscarinic acetylcholine receptor (E. Grzek, et al., folia medica copernicana 2, 98-101 (2014)). As shown in FIG. 9F, arterial SMCs in arterial conduits exhibited the expressions of M3 muscarinic acetylcholine receptor. The responses to acetylcholine (10 μM) were then examined in pre-constricted arterial conduits induced by phenylephrine. The application of acetylcholine indeed relaxed phenylephrine-induced contractions of arterial SMCs leading to the dilation of the arterial conduits approximately to the original size (FIG. 9G-H). Thus, the bioprinted arterial SMCs within the arterial conduits displayed important physiological functions, i.e., vasoconstriction and vasodilation, in response to stimuli by vasoconstrictor and vasodilator.

### In Vitro, Ex Vivo, and In Vivo Applicability of (Bio)Printed Vascular Conduits

The coronavirus disease 2019 (COVID-19) pandemic caused by the infection with SARS-CoV-2, has significantly affected our lives since December 2019. SARS-CoV-2 binds to the angiotensin-converting enzyme 2 (ACE2) receptor, via spike glycoprotein, for entering the host cells (C. B. Jackson, et al., Nature Reviews Molecular Cell Biology 23, 3-20 (2022)). SARS-CoV-2 had infected more than 220 million people, causing over 6.2 million deaths across the globe. New SARS-CoV-2 variants have been continuously evolving due to mutations in the SARS-CoV-2 genome, some of which are classified as variants of concern as they are more aggressive, highly transmissible, vaccine-resistant, and cause more-severe disease manifestations as compared to the original SARS-CoV-2 strain. In addition, multiple reinfections and relapses with SARS-CoV-2 have been recorded (S. K. Abrokwa, et al., PloS one 16, e0261221 (2021)). Notably, even though COVID-19 is primarily considered a respiratory disease, it can affect several other vital organ systems, including cardiovascular, renal, and brain systems. For example, recently, clinical observations and in vivo animal studies suggested that the adverse effects of COVID-19 on multiple organs are due to increased vascular dysfunction such as leaky vascular barrier and enhanced expression of von Willebrand factor that caused increased coagulation, cytokine release, and inflammation (L. C. Barbosa, et al., Vascular Pharmacology 137, 106829 (2021)).

3D-(bio)printed blood vessels that recapitulate key features of native blood vessels can be utilized as reliable preclinical in vitro models to study the direct vascular responses to the SARS-CoV-2 infections. We thus assessed the applicability of (bio)printed conduits as 3D vascular models for examining SARS-CoV-2 infection using pCoV-VPs and treatment efficacies of antiviral drugs using two clinically approved antiviral drugs, RMD and ADQ, that have been given to COVID-19 patients (A. J. Pruijssers, et al., Cell Reports 32, 107940 (2020)). Blood vessels are prone to SARS-CoV-2 infections because the ACE2 receptor is expressed by all vascular structural cells, including ECs, SMCs, fibroblasts, and pericytes, among other cell types (A. G. Harrison, et al., Trends Immunol 41, 1100-1115 (2020)).

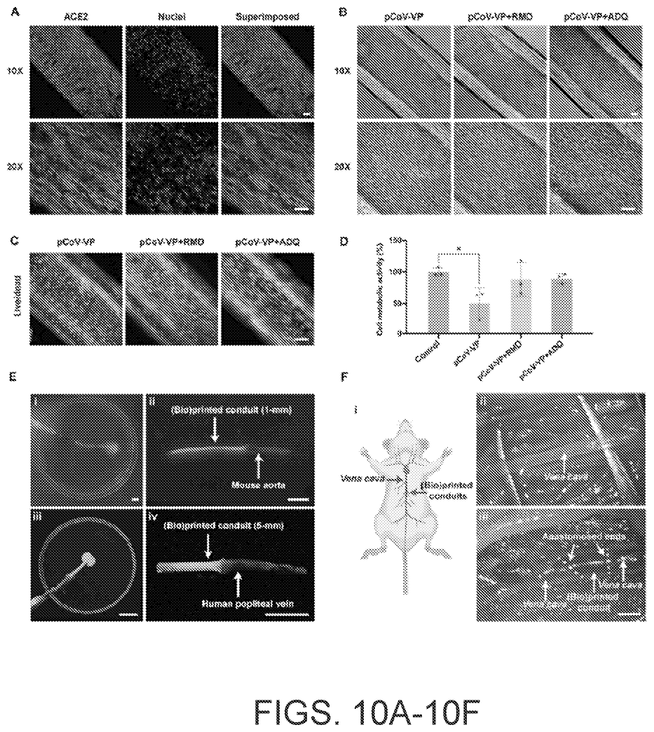

Prior to pCoV-VP infection, the expression of ACE2 receptor was assessed for the venous conduits. As shown in FIG. 10A, HUVSMCs within the venous conduits exhibited a high level of expression of the ACE2 receptor. The venous conduits were inoculated with mCherry-labeled pCoV-VPs at the multiplicity of infection of 0.5 for 48 h to identify the virus susceptibility in vitro in the presence of antiviral drugs. The conduits infected with pCoV-VPs in the absence of drugs were utilized as the control. After 48 h of exposure, the infection of cells with pCoV-VPs could be observed under fluorescence microscopy (FIG. 10B). The number of pCoV-VPs in the infected venous conduits was quantified by measuring luciferase activity. The viral entry in untreated venous conduits was set as 100%. The infection of venous conduits with pCoV-VPs was observed to be reduced by 38.3% in the presence of RMD and 73.2% in the presence of ADQ. Similarly, the cytopathic effect of pCoV-VPs was analyzed by live/dead assay (FIG. 10C) and (3-(4,5-dimethylthiazol-2-yl)-5-(3-carboxymethoxyphenyl)-2-(4-sulfophenyl)-2H-tetrazolium (MTS) assay (FIG. 10D). The cytopathic effects of pCoV-VPs were decreased in the presence of the antiviral drugs while improving the cell viability values and metabolic activities. Thus, these therapeutics significantly inhibited the viral entry- and infection-induced cell-death by the pCoV-VPs expressing the SARS-CoV-2 spike proteins.

Finally, to explore the future translational potential as vascular grafts, the (bio)printed conduits of different diameters were connected to native vessels. We initially (bio)printed a tube with a diameter of 1 mm and glued it to an ex vivo explanted mouse aorta to achieve anastomosis. Orange fluorescent beads were perfused from one open end of the (bio)printed conduit to pass the joining point and exit from the open end of the mouse aorta. As demonstrated in FIG. 10E-i, ii, the dye went through the entire leak-free lumen smoothly. To enhance this concept, we subsequently employed a freshly isolated popliteal vein harvested from a human patient receiving bypass surgery to connect with a (bio)printed larger conduit (5 mm in diameter). Similarly, no leakage was observed during the perfusion of green fluorescence beads from the natural vein to the (bio)printed vascular conduit (FIG. 10E-iii, iv). To this end, we conducted a proof-of-concept test in vivo in mice (FIG. 10F-i), where the vena cava was exposed (FIG. 10F-ii), followed by gluing a sterile (bio)printed vascular conduit to it from both ends. After the release of the clamps, the bloodstream was observed to flow through the vena cava to the (bio)printed conduit and to vena cava quickly without noticeable leakage (FIG. 10F-iii). This set of results illustrated that the (bio)printed conduits could be anastomosed to native vessels in various scenarios, direct evidence for potential in vivo vascular reconstruction applications, though the need for further optimization is acknowledged.

In summary, we developed DN tough hydrogel (bio)inks consisting of gelatin (or GelMA) and alginate with suitable rheological properties and cell-benign crosslinking for microfluidic (bio)printing of engineered small-diameter vascular conduits. The bioprinted venous and arterial conduits, consisting of the inner endothelial layer and outer smooth muscle layer, mimicked important features of the native veins and arteries, respectively. They exhibited superior mechanical properties, including burst pressure, elasticity, stretchability, and stiffness, comparable to those of the native vessels. In addition to the perfusability and selective permeability, expressions of relevant biomarkers were observed in the bioprinted conduits. Critically, the compact endothelial mono-layer provided barrier function, and the thicker smooth muscle layer of arterial conduits allowed the conduits to constrict and dilate similar to native arterioles. The (bio)printed vascular conduits could also serve as good in vitro vascular models to study vascular responses to viral infection and the efficacies of antiviral drugs. In addition, these (bio)printed conduits revealed the potential to be used as vascular grafts for in vivo applications. However, our technology is not without limitations. For example, despite enhanced high toughness, suturing these hydrogels tubes was found not easy due to their insufficient suture-retention abilities. Additional efforts are being devised to further improve our (bio)ink formulations. Moreover, comprehensive in vivo evaluations remain to be conducted.

### Materials and Methods

### Materials

This study did not generate new unique reagents. Gelatin from porcine skin (type-A, 300 bloom), methacrylic anhydride, sodium alginate (71238, medium-viscosity), sodium alginate (A1112, low-viscosity), FITC-Dex (3-5 kDa and 150 kDa), phenylephrine, acetylcholine, CaCl₂), bovine serum albumin (BSA), triton X-100 and EDTA were obtained from Sigma-Aldrich (Burlington, MA, USA). Paraformaldehyde (16%) was purchased from Electron Microscopy Sciences (Hatfield, PA, USA). mTG was purchased from Ajiomoto North America. Inc (Fort Lee, NJ, USA). Fetal bovine serum (FBS), DPBS, Dulbecco's modified Eagle medium (DMEM), trypsin-EDTA, 2-[4-(2-hydroxyethyl) piperazin-1-yl]-ethane sulfonic acid (HEPES buffer, 25 mM, pH 7.4), and antibiotic-antimycotic solution stabilized (Anti-Anti, 100×) were from Life Technologies (Carlsbad, CA, USA). 4′,6-diamidino-2-phenylindole (DAPI), Live/Dead® Viability/Cytotoxicity Kit, Alexa Fluor® 594-phalloidin, Alexa Fluor®488-phalloidin, dialysis membrane (Mw cutoff: 12,000-14,000 Da), and rabbit α-1a adrenergic receptor antibody was obtained from Thermo Fisher Scientific (Cambridge, MA, USA). Endothelial growth medium (EGM-2) and endothelial growth supplements were obtained from Lonza (Walkersville, MD, USA), and the smooth muscle cell growth medium-2 (SmGM-2) along with growth supplements was obtained from PromoCell (Heidelberg, Germany). Rabbit anti-ZO-1 antibody, rabbit anti-vascular endothelial (VE)-cadherin antibody, mouse anti-α-SMA antibody, rabbit muscarinic acetylcholine receptor antibody, and Alexa Fluor®594- or 488-conjugated goat anti-rabbit or goat anti-mouse secondary antibodies were purchased from Abcam (Cambridge, MA, USA). Rabbit ACE2 antibody was purchased from Rockland (Limerick, PA, USA). RBCs collected in citrate, phosphate, dextrose, adenine-formula 1 (CPDA-1) were purchased from Research Blood Components (Watertown, MA, USA). Polydimethylsiloxane (PDMS) was purchased from Dow Corning Inc. (Midland, MI, USA). All other chemicals used in this study were obtained from Sigma-Aldrich (Burlington, MA, USA) unless otherwise mentioned.

### Animal and Human Tissue Collection

All animal-relevant experiments were conducted in accordance with protocols approved by the local Institutional Animal Care and Use Committee and complied with the Guide for the Care and Use of Laboratory Animals. Nine-week-old male C57BL/6J mice weighing 20-24 g were purchased from The Jackson Laboratory (Bar Harbor, ME). The animals were maintained on a 12-hour light/dark cycle, and they received water and standard chow ad libitum.

Discarded human vascular tissues were harvested under a local institutional review board approved protocol. The sample was stored at −80° C. until use.

### Synthesis of GelMA

GelMA was synthesized following the previously described protocol we introduced (H. Ravanhakhsh, et al., Matter 5, 573-593 (2022)). Briefly, 10.0 g of type-A gelatin from porcine skin was added into 100 mL of DPBS and dissolved at 50° C. under a magnetic stirrer for 30 min. Then, 5.0 mL of methacrylic anhydride was added dropwise to the gelatin solution and kept stirring at 50° C. for 3 h. The reaction was quenched by 100 mL of warm DPBS (40° C.). Next, the reaction product was dialyzed against distilled water at 40° C. for 5 days using a dialysis membrane (M_{w} cutoff: 12,000-14,000 Da). Finally, the solution was filtered by a 0.2-μm filter and lyophilized to yield a white porous foam, which was stored at −20° C. for further use.

### Hydrogel Film Fabrication

Pure alginate hydrogel was obtained by ionic crosslinking of 1% alginate solution using a 2% CaCl₂) solution. Liquid-form hydrogel-precursors of 15% gelatin and hybrid (bio)ink containing 1% alginate and 15% gelatin were sandwiched between two glasses by 0.3-mm plastic spacers. After cooling down 4° C. for 2-3 min, the obtained physical gel was transferred to CaCl₂/mTG solution bath. After curing at room temperature for 30 min, the hydrogel was further cured at 37° C. overnight to obtain hydrogel films. To fabricate ionic and covalent single-network hydrogels, the crosslinker bath was switched to 2% CaCl₂ and 2% mTG, respectively. The dual-crosslinked hydrogels were also treated by the 3% EDTA to selectively remove the ionic crosslinks in the DN hydrogels.

### Rheology Tests

Rheology measurement was conducted on a rheometer (DHR-3, TA Instruments, New Castle, DE, USA) using a 1° core plate geometry with a diameter of 40 μm and a gap height of 26 μm. Apparent viscosities as a function of shear rate (0.01-200 s⁻¹) were measured via steady-state flow-sweep. Temperature-variant apparent viscosities were measured from 15 to 37° C. at a constant shear rate of 1 s⁻¹. Amplitude-sweep was measured as a function of shear strain (0.01-2,000%) via oscillation experiments using a fixed frequency of 1 Hz at constant temperatures.

### Microfluidic Extrusion (Bio)Printing of Acellular Mono-Layered and Dual-Layered Conduits

We used two types of coaxial extrusion systems with two or three channels to (bio)print venous and arterial conduits, respectively. To achieve broadly geometry-tunable printability and reassemblability benefiting recycling of the nozzles, we first designed (bio)printing nozzle systems (BNS) in a computer-aided design (CAD) software (SolidWorks, Dassault Systemes, Vélizy-Villacoublay, France). Then, clear resin (green Translucent UV resin, Anycubic, Shenzhen, China) was used to print all the pieces of the BNS by a commercial stereolithography apparatus (SLA) printer (Anycubic Photon), which granted great versatility in designs and very fast fabrication times. The nozzles were assembled using commercial screws and bolts and were put together with silicon tubes using commercial blunt needles. The BNS could be easily disassembled to wash or switch every piece.

BNS with two coaxial channels were used to (bio)print mono-layered acellular tubular conduits (FIG. 7A) using the (bio)ink composed of medium-viscosity alginate (0.5, 1, and 2%) and gelatin (10, 15, and 20%). 2% CaCl₂ solution flowing in the core layer acted as the crosslinker for alginate in the hybrid (bio)ink (FIG. 7A). (Bio)inks containing 2% low-viscosity alginate and gelatin or GelMA were also used to (bio)print tubes for mechanical measurement and physiological stability study. The (bio)printed tubes were later crosslinked with CaCl₂/mTG overnight resulting in hollow mono-layered acellular conduits with DN hydrogel walls. BNS with three coaxial channels were used to (bio)print dual-layered acellular tubular conduits.

### Mechanical Tests

The tensile and pure-shear tests for film and tube samples were performed on a universal tensile machine (Instron 3324, Norwood, MA, USA) with a load cell capacity of 100 N. Rectangular samples with 0.3-0.4 mm of thickness and 6 mm in width were cut from the film samples for mechanical measurements. For the pure-shear test, a precut with a length of 2 mm was introduced in the middle. (Bio)printed tubes with a diameter of 800-900 μm and thickness of 200-300 μm were also used for the tensile test. The gauge length for all the samples was set at approximately 8 mm, and the stretching rate was fixed at 0.5 min⁻¹. In loading-unloading test, the film samples were first loaded to 25% strain and unloaded using the same stretching rate. Successive loading-unloading was also conducted using increasing applied maximum strains. The nominal stress was defined as the applied force divided by the cross-sectional area in the undeformed state. The strain was defined as the elongated sample length divided by the initial length. The tensile modulus was determined by the slop of the stress-stretch curve within the 3% strain. The toughness and hysteresis ratio of hydrogels were calculated according to literature (C. Xiang, et al., Materials Today 34, 7-16 (2020)). The fresh native blood vessels, including the mouse vena cava (approximately 800 μm of outer diameter and 110 μm of thickness) and aorta (approximately 1,200 μm of diameter and 180 μm of thickness) with similar size to the (bio)printed acellular conduits and were harvested.

### Measurement of Burst Pressures

Hollow tubes of different sizes were fabricated, and the mouse vena cava and aorta were used as controls. The acellular vascular conduits were highly inflatable and able to be tied to metal needles of the bioreactor by surgical knots (5-0 sutures). The two ends of each tube were fixed onto two blunt needles. Then, it was well-inflated by pressured air without leakage. To quantify the burst pressure, one end of each sample was closed by suture, while the other end was cannulated to a blunt needle connecting to a gas tank and a pressure-sensor. Burst pressure was measured by pressurizing the sample with compressed air until failure and calculated considering zero external pressure and no axial loading.

### Permeability and Diffusion Tests

To evaluate the perfusability, a narrow but long mono-layered tube (800 μm in diameter and 25 cm in length) was (bio)printed and randomly coiled in a vial filled with deionized water, leaving the two ends outside, followed by perfusing RBC-suspension from one of the ends.

To evaluate the barrier performances of the (bio)printed conduits, a PDMS mold with one or two culture medium reservoirs was fabricated, and two or four blunt needles matching the investigated tube size were inserted into the two sides of each reservoir, in which the medium was filled. Sequentially, a (bio)printed vascular conduit was fixed between the two needles by surgical knots. Then, the conduit-connected bioreactor, further linked to a peristaltic pump (Elemental Scientific, Omaha, NE, USA) which was loaded by 3-5-kDa or 150-kDa FITC-Dex. During permeability characterizations, all parameters of the circulation system and the fluorescence microscope settings between groups, including perfusion rate, fluorescence dye concentration, tube length, tube size, medium volume, exposure time, and brightness threshold, remained unchanged.

To explore the selective permeability for small and large molecules, we chronologically imaged the fluorescence of the tubes at predefined time points of 10 min, 4 h, 8 h, and 24 h, and measured the fluorescence intensity profiles across the central portion of the reservoir in these images by the method reported in the literature (S. Massa, et al., Biomicrofluidics 11, 044109 (2017)). To quantify the difference, diffusional permeability was calculated according to an equation published (D. B. Kolesky, et al., Proc Natl Acad Sci USA 113, 3179-3184 (2016)):

$${P}_{d} = \frac{1}{{l}_{1} - {l}_{b}} ( \frac{{l}_{2} - {l}_{1}}{t} ) \frac{d}{4}$$

where P_{d} is the diffusional permeability coefficient, l₁ is the average intensity at the initial time point, l₂ is the average intensity at the given time (t, approximately 30 min), l_{b} is the background intensity (before introducing FITC-Dex), and d is the channel diameter. The measurements are performed on the (bio)printed conduits with and without endothelium. To verify the barrier function of the endothelium of the cell-seeded (bio)printed vascular conduits, the fluorescence images and videos were captured in the first 10 min since the onset of perfusion. They were subsequently processed into greyscale images in ImageJ (National Institutes of Health, Bethesda, MD, USA) followed by calculating the fluorescence area ratio of each image, which was defined as the diffusion rate, according to the method published (D. Wang, et al., Journal of investigative surgery: the official journal of the Academy of Surgical Research 34, 393-400 (2021)).

### Cell Culture

Primary HUVSMCs were purchased from ScienCell Research Laboratories (Carlsbad, CA, USA), whereas primary HUVECs were obtained from Lonza (Walkersville, MD, USA). Primary HUASMCs and primary HUAECs were obtained from PromoCell. Both HUVECs and HUAECs were cultured EGM-2 medium supplemented with endothelial growth supplements and 1% (v/v) anti-anti. Similarly, both HUVSMCs and HUASMCs were cultured in SmGM-2 supplemented with growth supplements and 10% (v/v) FBS and 1% (v/v) anti-anti. The cells were incubated at 37° C. and 5% CO2 in a 95% humidified cell incubator until 70-80% confluency. The respective culture medium was changed every 3^{rd} day.

### Cell Seeding in the (Bio)Printed Mono-Layered Tubular Conduits

The HUVSMCs at 70-80% confluence were trypsinized, centrifuged, and resuspended in medium at 8×10⁶ viable cells mL⁻¹. PDMS-wax (95:5%) mold with multiple straight channels were fabricated. Approximately 100 μL of HUVSMC-suspension was first loaded into channels and end-closed mono-layered tubes were placed in the channels individually containing the HUVSMC-suspension, followed by pouring additional 100 μL of HUVSMC-suspension on the top surfaces of tubes in the channels. After 1 day of incubation at 37° C. in the incubator, HUVSMCs were found to be selectively attached to the outer surfaces of the tubes rather than the mold surfaces due to the pronounced hydrophobicity of the latter. On day 3 of HUVSMC seeding, HUVECs were seeded in the lumen by perfusing 100-150 μL of HUVEC-suspension at 1×10⁷ viable cells mL⁻¹. After culturing of the conduits at 37° C. for an additional 7 days (a total of 10 days after seeding HUVSMCs), hollow venous conduits, with a compact layer of endothelium covering the inner wall of the lumen and a thin, smooth muscle sheath across the outer surface, were eventually formed for downstream studies.

### Microfluidic Extrusion Bioprinting of Dual-Layered Cellular Conduits

BNS with three coaxial channels were also used to bioprint dual-layered cellular tubular conduits. HUASMCs at 70-80% confluence were trypsinized, centrifuged, and resuspended in bioink at 3×10⁷ viable cells mL⁻¹. While MAlg1Gel15 was used for (bio)printing the inner layer, the LAlg2GM3 bioink was selected for the encapsulation and bioprinting of the outer HUASMC-layer so as to facilitate growth and proliferation of these cells. 2% CaCl₂ solution flowing in the core facilitated the crosslinking of alginate in the bioink (FIG. 7A). The bioprinted dual-layered tubular conduits were later transferred into CaCl₂/mTG crosslinker prepared in SmGM-2 culture medium. After 12 h of incubation at 37° C. in the incubator, the crosslinker solution was replaced with fresh SmGM-2 culture medium. At 14 days of bioprinting, HUAECs were seeded in the lumen by perfusing 100-150 μL of HUAEC-suspension at 1×10⁷ viable cells mL⁻¹. After incubation of arterial conduits at 37° C. for an additional 7 days (thus a total of 24 days after bioprinting HUASMCs), hollow arterial conduits with a compact layer of endothelium covering the inner lumen wall and a thick smooth muscle sheath throughout the outer surface were eventually formed for downstream studies.

### Cell Viability Assay

Cell viability was measured by the Live/Dead® viability/cytotoxicity kit according to the manufacturers' instructions. Briefly, the vascular conduits were washed with DPBS twice and placed in the wells of a 6-well plate, followed by the addition of live/dead staining solution containing 1-μL mL⁻¹ calcein-AM (4 mM) and 2-μL mL⁻¹ ethidium homodimer-1 (2 mM) in DPBS. After incubation at 37° C. for 30 min, the samples were washed three times with DPBS and observed under an Eclipse Ti2 inverted microscope (Nikon, NY, USA). Percentages of viable cells were determined using the ImageJ software.

### F-Actin Staining

For morphological analyses, Alexa Fluor® 488-phalloidin or Alexa 549-phalloidin was used for F-actin staining. The vascular conduits were washed with deionized water twice and fixed with 4% (v/v) paraformaldehyde for 15 min. After gentle washing for three times, the samples were permeabilized with 0.1% (v/v) Triton X-100 for 1 h at room temperature. Alexa 488-phalloidin or Alexa 549-phalloidin (1:200 (v/v) in 0.1% BSA) was added to the samples and incubated for t h at room temperature. The samples were washed again with deionized water and then stained with the DAPI (1:1000 (v/v) in DPBS) for 5 min at room temperature. After washing three times with deionized water, fluorescence images were taken using a Zeiss LSM880 confocal microscope (Zeiss, NY, USA).

### Immunostaining

The biofunctionalities of the endothelial and smooth muscle layers in vein, and arterial conduits were further confirmed by immunostaining of the cells for cell-specific markers including ZO-1, laminin, α-SMA, and tubulin. The vascular conduits were fixed with 10% (v/v) formalin for 30 min at room temperature. After washing three times with deionized water, the samples were incubated with the permeabilization buffer (0.1% (v/v) Triton X-100 in deionized water) for 1 h and blocked with 5% (v/v) goat serum in deionized water for 2 h at room temperature. The samples were then incubated with the desired primary antibody (1:200 dilution in blocking buffer) overnight at 4° C. The samples were washed three times with deionized water and incubated overnight at 4° C. with the relevant secondary antibody (Alexa Fluor® 594-conjugated goat anti-rabbit secondary antibody or Alexa Fluor® 488-conjugated goat anti-mouse secondary antibody) at 1:200 dilution in blocking buffer. Finally, the nuclei were counterstained with DAPI after washing with deionized water and examined under the Zeiss LSM880 confocal microscope.

### Vasoactivity Assay

Phenylephrine-induced vasoconstriction and acetylcholine-induced vasodilation were assessed on arterial conduits. The drugs at given concentrations were prepared in the physiological salt solution composed of 123-mM NaCl, 5-mM KCl, 15.5-mM NaHCO₃, 1.2-KH₂PO₄ mM, 1.2-mM MgCl₂, 1.25-mM CaCl₂), 11-mM D-glucose, and 25-mM HEPES at pH 7.40 adjusted with NaOH. Arterial conduits were first perfused with 10-μM phenylephrine to assess vasoconstriction responses. After 5 min, the vasodilation responses were assessed by adding 10-μM acetylcholine to the precontracted arterial conduits induced by phenylephrine. Phenylephrine-induced contraction and acetylcholine-induced dilation were examined under observed under the Eclipse Ti2 inverted microscope, and percentages of changes in the diameter were determined using ImageJ.

pCoV-VPs Production and Infection of Vascular Conduits

Production of pCoV-VPs was carried out as described in our previous study (D. Huang, et al., Proc Natl Acad Sci USA 118, (2021)). Briefly, HEK293T cells (5×10⁵ cells well⁻¹) were cultured in 6-well plates for 24 h of at 37° C. in the incubator and treated with 1.0-μg of pCMV3-SARS-CoV2-Spike (Sino Biological, Chesterbrook, PA, USA), 1.0-μg of pNL4-3 mCherry Luciferase (Addgene, MA, USA), and 0.5 μg of pAdvantage (Promega, Madison, WI, USA) using the TransIT-X2 transfection reagent (Abcam, Waltham, MA, USA) to produce pCoV-VPs according to the manufacturer's instructions. After 48-h, the supernatants containing pCoV-VPs were collected, centrifuged at 10,000 g for 5 min to remove cell debris, and concentrated using a poly(ethylene glycol) virus precipitation kit (Abcam, Waltham, MA, USA) according to the manufacturer's instructions. pCoV-VPs were stored at 80° C. until use.

To investigate pCoV-VP infection on vascular conduits and effects of model antiviral drugs, the samples were infected with pCoV-VPs at multiplicity of infection of 0.5 in the presence of remdesivir (10 μM, MedKoo Biosciences, Morrisville, NC, USA) or amodiaquine (10 μM, Sigma-Aldrich, Burlington, MA, USA)). After 48 h of infection, the mcherry positive cells, which reflect the pCoV-VPs infections, were observed under the Eclipse Ti2 inverted microscope. Similarly, luciferase activity, which reflects the number of pCoV-VPs in the host cells, was measured after 48 h of post-infection using the Luciferase assay system (Promega, Madison, WI, USA) according to the manufacturer's instructions. The infection values were calculated from the intensities measured for drug-treated samples divided by the average intensity measured for the control sample and multiplied by 100%.

The cytopathic effect of pCoV-VPs infections was assessed by the live/dead assay and the MTS assay using the CellTiter 96© AQueous one solution cell proliferation assay kit (Promega, Madison, WI, USA) according to the manufacturer's instruction. Uninfected samples served as controls.

### Ex Vivo and In Vivo Anastomose Tests

For ex vivo connection tests, a (bio)printed small hydrogel conduit (1 mm in diameter) was anastomosed to a piece of mouse aorta by dropping fast-curing adhesive droplets (Newell Brands Inc., GA, USA) on the interface, while a larger conduit (5 mm in diameter) was connected to a piece of the human popliteal vein. The (bio)printed hydrogel conduits anastomosed with native mouse or human blood vessels were then perfused with fluorescent microbeads suspended in DPBS.

For in vivo anastomose test, mice were anesthetized by isoflurane inhalation (5% induction, 2% maintenance). After careful incision and dissection, the vena cava was exposed, followed by closing the distal end and proximal end of the vein with vascular clamps, and the 2.5-cm segment was isolated to be cross-resected. A sterile (bio)printed vascular conduit was inserted into and stuck with the two exposed ends by adhesive glue. Clamps were then released and inspected for the flow of the blood through the anastomosed vascular conduits.

### Statistical Analyses

All the experiments were done in triplicates to quintuples for each data point and were repeated three to five times. Results were expressed as means±standard errors of the means (SEMs) of three independent experiments, and statistical comparisons were performed using the one-way analysis of variance (ANOVA). p<0.05 was considered statistically significant.

### Example 3: Triple Co-Axial Printing of Double Layer Tubes Including Polyurethane

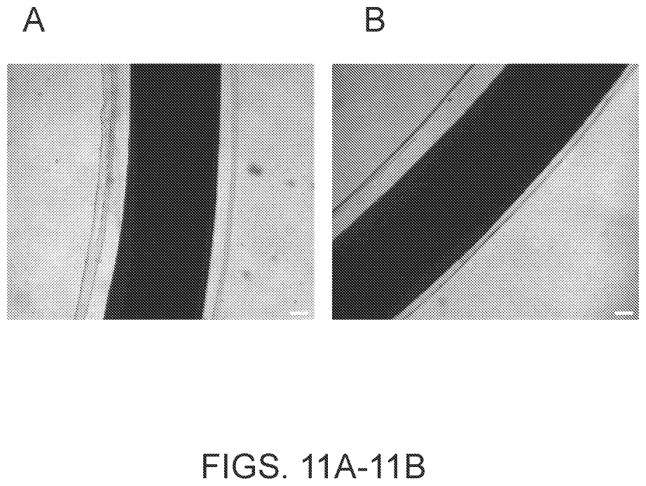

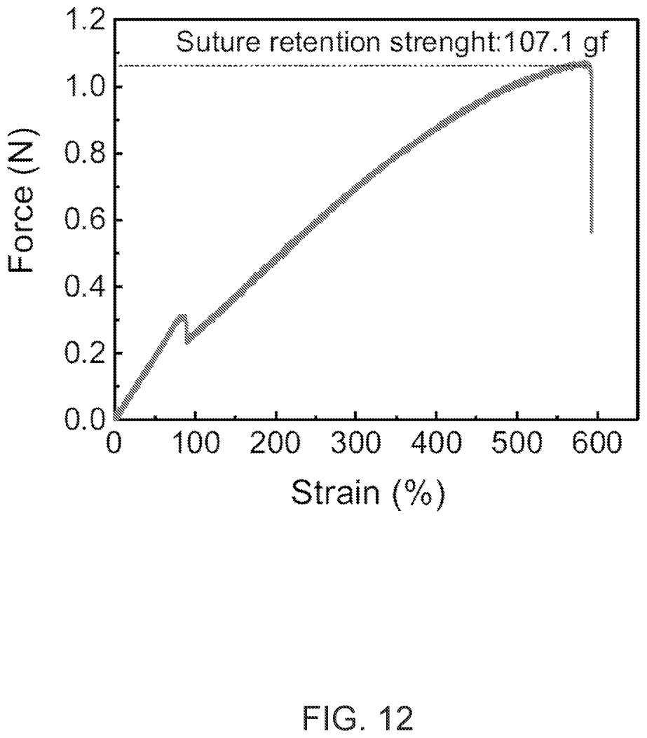

The inventors have also used coaxial printing to create hollow tubes including polyurethane in one of the layers. The tubes prepared had an outer diameter of about 4-5 mm using triple coaxial printing of double layers. A cross-section view of the double layer tubes is provided by FIGS. 11A and 11B. The inner layer of the tubes comprises 20% polyurethane by weight and 5% GelMA by weight, while the outer layer of the tubes comprises 2% alginate by weight and 10% gelatin by weight. A suture retention test was carried out for a propylene 4-0 suture. The results are shown in FIG. 12, which shows that the polyurethane-containing hollow tubes exhibited a suture retention strength of 107.1 gf. A cyclic strain retention test using a maximum of 25% strain and a stretch rate of 48 cycles/min again showed retention of propylene 4-0 sutures.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A 3D-printed biomaterial, comprising a double-networked hydrogel comprising gelatin or gelatin methacryloyl (GelMA), alginate, and cells.

2. The 3D-printed biomaterial of claim 1, wherein the hydrogel comprises a stronger layer having a higher stiffness and a smaller mesh size, and a weaker layer having a lower stiffness and a larger mesh size.

3. The 3D-printed biomaterial of claim 2, wherein the weaker layer comprising 1% to 3% alginate by weight and 2% to 4% GelMA by weight, and the stronger layer comprising 0.5% to 2% alginate by weight and 10% to 20% GelMA by weight.

4. The 3D-printed biomaterial of claim 2, wherein the weaker layer comprises cells.

5. The 3D-printed biomaterial of claim 2, wherein the stronger layer has a Young's modulus from about 50 to about 1000 kPa.

6. The 3D-printed biomaterial of claim 1, wherein the 3D-printed biomaterial comprises a first layer comprising the double-networked hydrogel, and a second layer comprising a mixture of GelMA and polyurethane.

7. The 3D-printed biomaterial of claim 1, wherein the double-networked hydrogel comprises a bi-continuous emulsion.

8. The 3D-printed biomaterial of claim 7, wherein the double-networked hydrogel further comprises polyvinyl alcohol (PVA) or polyethylene oxide (PEO).

9. The 3D-printed biomaterial of claim 1 wherein the gelatin is gelatin methacryloyl (GelMA).

10. The 3D-printed biomaterial of claim 1, wherein the biomaterial comprises a hollow tube.

11. The 3D-printed biomaterial of claim 10, wherein the hollow tube comprises a middle layer comprising the double-networked hydrogel, an inner layer comprising endothelial cells, and an outer layer comprising smooth muscle cells.

12. The 3D-printed biomaterial of claim 11, wherein the middle layer has a thickness from 50 μm to 500 μm.

13. The 3D-printed biomaterial of claim 11, wherein the hollow tube is an artery.

14. The 3D-printed biomaterial of claim 13, wherein middle layer comprises an upper middle layer comprising 1% to 3% alginate by weight and 2% to 4% GelMA by weight, and a lower middle layer comprising 0.5% to 2% alginate by weight and 10% to 20% GelMA by weight.

15. The 3D-printed biomaterial of claim 11, wherein the hollow tube is a vein.

16. The 3D-printed biomaterial of claim 13, wherein the alginate comprises 0.5% to 2% by weight and the gelatin comprises 10% to 20% by weight.

17. The 3D-printed biomaterial of claim 11, wherein the biomaterial exhibits vasoactivity.

18. The 3D-printed biomaterial of claim 1, wherein the alginate has a molecular weight from about 30 kDa to about 300 kDa.

19. The 3D-printed biomaterial of claim 1, wherein the biomaterial is a scaffold.

20. The 3D-printed biomaterial of claim 1, wherein the biomaterial is cartilage.

21. A method of making a 3D-printed biomaterial, comprising
extruding a hydrogel comprising gelatin or GelMA and alginate from a 3D-printer;
converting the hydrogel to a double-networked hydrogel by
a) cross-linking the alginate with a first cross-linking method; and
b) cross-linking the gelatin or GelMA with a second cross-linking method.

22. The method of claim 21, wherein the hydrogel extruded by the 3D-printer comprises cells.

23. The method of claim 21, wherein the alginate comprises 0.5% to 2% by weight and the gelatin comprises 10% to 20% by weight of the hydrogel.

24. The method of claim 21, wherein the hydrogel comprises a stronger layer having a higher stiffness and a smaller mesh size, and a weaker layer having a lower stiffness and a larger mesh size.

25. The method of claim 24, wherein the stronger layer has a Young's modulus from about 50 to about 1000 kPa.

26. The method of claim 24, wherein the weaker layer comprising 1% to 3% alginate by weight and 2% to 4% GelMA by weight, and the stronger layer comprising 0.5% to 2% alginate by weight and 10% to 20% GelMA by weight.

27. The method of claim 21, further comprising viscoelastic phase-separation of the hydrogel to form a bi-continuous emulsion.

28. The method of claim 21, wherein the first cross-linking method comprises using calcium chloride and the second cross-linking method comprises using calcium transglutaminase.

29. The method of claim 21, wherein the extruded hydrogel comprises a scaffold.

30. The method of claim 21, wherein the step of extruding a hydrogel further comprises forming a hollow tube having an inner surface and an outer surface using a coaxial extrusion 3D printing method.

31. The method of claim 30, the hydrogel comprises a stronger layer adjacent to the inner surface having a higher stiffness and a smaller mesh size, and a weaker layer adjacent to the outer surface having a lower stiffness and a larger mesh size.

32. The method of claim 30, further comprising seeding a surface of the hollow tube with cells.

33. The method of claim 30, further comprising seeding the outer surface of the hollow tube with smooth muscle cells and seeding the inner surface of the hollow tube with endothelial cells.

34. The method of claim 21, wherein the alginate has a molecular weight from about 30 kDa to about 300 kDa.

35. A method of treating a damaged or diseased blood vessel in a subject, comprising attaching a 3D printed blood vessel to the damaged or diseased blood vessel of the subject and rerouting blood flow so that blood flows through the 3D printed blood vessel, wherein the 3D-printed blood vessel comprises a hollow tube comprising a middle layer comprising a double-networked hydrogel comprising gelatin and alginate, an inner layer comprising endothelial cells, and an outer layer comprising smooth muscle cells.

36. The method of claim 35, wherein the damaged or diseased blood vessel is an atherosclerotic blood vessel.

37. The method of claim 35, wherein the damaged or diseased blood vessel is an artery.

38. The method of claim 37, wherein the damaged or diseased blood vessel is a coronary artery.

39. The method of claim 35, wherein the damaged or diseased blood vessel is a vein.

40. A method of testing a vasoactive drug, comprising contacting a 3D-printed blood vessel with an effective amount of a vasoactive drug, and observing the effect of the vasoactive drug on the 3D-printed blood vessel, wherein the 3D-printed blood vessel comprises a hollow tube comprising a middle layer comprising a double-networked hydrogel comprising gelatin and alginate, an inner layer comprising endothelial cells, and an outer layer comprising smooth muscle cells.

41. The method of claim 40, wherein the vasoactive drug is a vasoconstricting agent.

42. The method of claim 40, wherein the vasoactive drug is a vasodilating agent.

43. The method of claim 40, wherein the 3D printed blood vessel is a vein and wherein the middle layer comprises 0.5% to 2% alginate by weight and 10% to 20% GelMA by weight.

44. The method of claim 40, wherein the 3D-printed blood vessel is an artery and wherein the middle layer comprises an upper middle layer comprising 1% to 3% alginate by weight and 2% to 4% GelMA by weight, and a lower middle layer comprising 0.5% to 2% alginate by weight and 10% to 20% GelMA by weight.
